(19) United States
(12) Patent Application Publication
(10) Pub. No.: US 2014/0286964 A1
(43) Pub. Date: Sep. 25, 2014

(54) Methods for Identifying Janus Kinase (JAK) Modulators for Therapeutics

(71) Applicant: NEW YORK UNIVERSITY, New York, NY (US)

(72) Inventors: Stevan R. Hubbard, New York, NY (US); Rajintha M. Bandarayanake, Norwalk, CT (US); Olli Silvennoinen, Tampere (FI); Daniela Ungureanu, Tampere (FI)

(21) Appl. No.: 14/353,332

(22) PCT Filed: Oct. 24, 2012
(86) PCT No.: PCT/US2012/061517

§ 371 Date: Apr. 22, 2014

Related U.S. Application Data
(60) Provisional application No. 61/550,554, filed on Oct. 24, 2011.

(51) Int. Cl. G06F 19/16; G01N 33/50; C07K 16/40; G01N 33/573

(52) U.S. Cl. CPC G06F 19/16; G01N 33/573; G01N 33/5041; C07K 16/40
USPC 424/158.1; 435/7.1; 506/9; 506/8; 530/389.3

(57) ABSTRACT

The present invention provides methods for designing agents such as ligands capable of binding to a Janus kinase (Jak), particularly a Jak2 JH2, and especially a JH2 V617F, screening methods for identifying agents such as ligands and small molecules capable of binding to the same, and computer assisted methods for designing and identifying such agents. Further, the present invention provides methods for treating myeloproliferative neoplasias (MPNs) along with agents such as ligands and small molecules identified or designed using the methods described.

### FIELD OF THE INVENTION

The present invention relates to the fields of molecular biology, and more particularly, to molecular signaling as well as screening assays.

### BACKGROUND OF THE INVENTION

Janus kinases (Jaks) are members of the non-receptor protein tyrosine kinase family and are key components of signaling pathways in cells of the immune system and in hematopoietic cells (Yamaoka, et al., Genome Biol. 2004; 5: 253; Ghoreschi, et al., Immunol. Rev. 2009; 228: 273-287). Jaks are bound to the cytoplasmic domains of cytokine receptors and, upon cytokine-mediated receptor dimerization, undergo autophosphorylation (in trans) on tyrosine residues, which stimulates their tyrosine kinase activity. Activated Jaks phosphorylate specific tyrosine residues on the cytokine receptors to which they are associated, which then serve as recruitment sites for Stats (signal transducers and activators of transcription). Recruited Stats are phosphorylated by Jaks, dimerize, and then translocate to the nucleus to serve as transcriptional regulators (FIG. 1). Lymphocyte development, proliferation and survival, as well as the initial responses of cells of the adaptive immune system, are entirely dependent upon signaling through the Jak-Stat pathway (Yamaoka, et al., Genome Biol. 2004; 5: 253; Levy, et al., Nat. Rev. Mol. Cell. Biol. 2002: 3: 651-662).

There are four mammalian members of the Jak family: Jak1-3 and Tyk2. These tyrosine kinases, which are approximately 125 kDa in size, possess four domains: a FERM (band 4.1/ezrin/radixin/moesin) domain, an SH2 (Src homology-2)-like domain, a pseudokinase domain (Janus homology-2 PH21), and a tyrosine kinase domain (JH1) (FIG. 2). Jak1, Jak2, and Tyk2 are ubiquitously expressed, whereas Jak3 is expressed primarily in hematopoietic cells. Each Jak interacts with a subset of cytokine receptors, with Jak2 mediating signaling by cytokines such as growth hormone, prolactin, erythropoietin, and interleukin-3 (Ghoreschi, et al., Immunol. Rev. 2009; 228: 273-287).

Extensive biochemical studies have established that: (i) the FERM domain is primarily responsible for the association of Jaks with cytokine receptors, (ii) the SH2-like domain does not function as a phosphotyrosine-binding domain (as do canonical SH2 domains), (iii) the pseudokinase domain negatively regulates the activity of the tyrosine kinase domain, and (iv) the tyrosine kinase domain is activated via trans-phosphorylation of tandem tyrosines in the activation loop (Y1007/Y1008 in Jak2) (Ghoreschi, et al., Immunol. Rev. 2009; 228: 273-287; Haan, et al. J. Cell. Mol. Med. 2010; 14: 504-527). In addition to Y1007/Y1008, several other sites of tyrosine and serine phosphorylation have been mapped in Jak2 (FIG. 2), which serve to regulate Jak2 catalytic activity, either positively or negatively (Ghoreschi, et al., Immunol. Rev. 2009; 228: 273-287) To date, high-resolution structural information is available for only the tyrosine kinase domains (JH1) of Jak proteins.

Deletion studies of the pseudokinase domain (JH2) demonstrated that JH2 negatively regulates Jak2 catalytic activity (JH1) to maintain the basal state (non-cytokine-stimulated), probably through a direct interaction between JH2 and the tyrosine kinase domain (JH1) (Saharinen, et al., Mol. Cell. Biol. 2000; 20: 3387-3395; Saharinen, et al., J. Biol. Chem. 2002; 277: 47954-47963). Although it is clear from these and other studies that JH2 functions as a negative regulator of JH1 activity, it is also evident that full activity of Jak2 requires an intact ΔJH2 and JH2 point mutants, in which the structural integrity of the domain is compromised, exhibit increased basal-level kinase activity, but the activity is not further increased by cytokine stimulation to the level of wild-type Jak2 (Saharinen, et al., J. Biol. Chem. 2002; 277: 47954-47963; Chen, et al., Mol. Cell. Biol. 2000; 20: 947-956).

JH2 had been previously classified as a pseudokinase because: (i) no tyrosine kinase activity apart from that of JH1 in Jaks had been observed, and (ii) sequence alignments revealed that several key catalytic residues conserved in active protein kinases have been substituted in JH2. These include an aspartic acid in the catalytic loop (N673 in Jak2), an arginine in the catalytic loop (K677 in Jak2), a phenylalanine in the activation loop (DFG motif, P700 in Jak2) and a glutamic acid in α-helix C (αC) (A597 in Jak2).

JH2 of Jak2 is actually a bona fide protein kinase (not a pseudokinase), phosphorylating a serine (S523) in the SH2-JH2 linker and a tyrosine (Y570) in JH2 (Ungureanu, et al., Nat. Struct. Mol. Biol. AOP, Aug. 14 (2011)). These residues had been shown previously to be negative regulatory sites in Jak2 (Ishida-Takahashi, et al., Mol. Cell. Biol. 2006; 26: 4063-4073; Feener, et al., Mol. Cell. Biol. 2004; 24: 4968-4978; Argetsinger, et al., Mol. Cell. Biol. 2004; 24: 4955-4967). These in vitro and in-cell data demonstrate that the catalytic activity of JH2 is critical for maintaining a low basal level of Jak2 activity.

A large number of mutations in Jak genes have been mapped in patients with myeloproliferative neoplasias (MPNs), including polycythemia vera, primary myelofibrosis, essential thrombocythemia, acute lymphoblastic leukemia, and acute myeloid leukemia (Haan, et al. J. Cell. Mol. Med. 2010; 14: 504-527). The mutations in Jaks that give rise to these diseases render the enzymes constitutively active, and a majority of the mutations map to JH2 (pseudokinase domain) (Haan, et al. J. Cell. Mol. Med. 2010; 14: 504-527), including the most commonly mapped mutation, V617F in Jak2 (Kralovics, et al., N. Engl. J. Med. 2005; 352: 1779-1790; James, et al., Nature 2005; 434: 1144-1148). Several small-molecule Jak2 inhibitors are now in clinical trials for primary myelofibrosis (Pardanani, Leukemia 2008; 22: 23-30). Our structural and biochemical studies could lead to the design of inhibitors that are selective for the mutated forms of Jaks (e.g., Jak2 V617F), which should alleviate the common side effects of anemia and thrombocytopenia that result from inhibition of wild-type Jaks as well as the constitutively active mutants.

A structural understanding of the autoregulation of Jaks by the pseudokinase domain is one of the important unsolved problems in the field of tyrosine kinase signaling. This mechanistic problem is made even more compelling by our recent finding that JH2 is an active protein kinase. A crystal structure of JH2 will provide, along with supporting biochemical data, the molecular basis for the catalytic activity of JH2, and a crystal structure of JH2-JH1 will provide the mechanism by which JH2 autoinhibits JH1. Moreover, the crystal structures will allow rationalization of mutations in JH2 (e.g., V617F) that cause MPNs, and should facilitate the development of novel inhibitors to combat these diseases.

### SUMMARY OF THE INVENTION

In one aspect, the present invention provides methods for designing ligands capable of binding to a Janus kinases (Jak) to modulate catalytic activity and/or downstream signaling. In particular, the invention provides methods for designing ligands capable of selectively binding a Jak2 V617F.

In a second aspect, the invention provides screening methods for identifying agents such as small molecules capable of binding to a Janus kinase (Jak) such as a JH2 to modulate catalytic activity and/or downstream signaling. In particular, the invention provides methods for screening for molecules such as small molecules capable of selectively binding a Jak2 V617F. Modulators identified using the methods of the invention may be capable of inhibiting the catalytic activity and/or downstream signaling of the Janus kinase (Jak).

In one embodiment, the invention provides methods for identifying agents such as small molecules that bind selectively to Jak2 JH2 V617F. Such small molecules may be useful for treating such diseases as myeloproliferative neoplasias. In some instances, the three-dimensional coordinates (spatial) of Jak2 JH2, wild type (WT) and V617F, as given by the crystal structures, may be used for in silico (computer) screening to identify compounds that bind to JH2 V617F but not to JH2 WT. In such screening techniques, compounds contained in large libraries such as on the computer may be docked one-by-one to the target protein (JH2 WT or V617F). A score may be assigned to each compound-target combination. Those compounds having the best differential scores (V617F vs. WT) may be candidates as selective binders of JH2 V617F. These compounds may be obtained and tested in vitro or in vivo to determine their ability to bind differentially to JH2 V617F versus WT.

In another embodiment, the expressed and purified proteins, JH2 WT and V617F, may be used in high-throughput screening to identify agents such as small molecules that bind selectively to JH2 V617F. Such methods may incorporate standard procedures and methods well known in the art.

In a third aspect, the invention provides screening methods for designing or identifying agents such as small molecules capable of binding to both a Janus kinase (Jak) such as a JH2 wild type (WT) and a Jak2 JH2 V617F. Such agents may be useful to restore the JH2 wild-type conformation to a Jak2 JH2 V617F. Such agents may bind both a JH2 wild type (WT) and a Jak2 JH2 V617F with specificity. Such agents may be useful to reverse any constitutive activating affects of Jak2 JH2 V617F. As such, such agents may be useful for treating one or more myeloproliferative neoplasias (MPNs). Such agents may for instance bind an ATP-binding pocket of a JH2. These methods may be practiced in vitro or in silico and may be computer-assisted.

In a fourth aspect, the present invention provides three-dimensional structural information for a Janus kinases (Jak), particularly a Jak JH2, and more particularly a Jak2 JH2 V617F. Such structural information may be used advantageously in methods for designing agents such as small molecules (e.g., inhibitors) specific for a Janus kinase (Jak), particularly a Jak JH2, and more particularly a Jak2 JH2 V617F domain to modulate or inhibit catalytic activity and/or downstream signaling. In particular, such structural information may be useful in methods for designing agents such as small molecules capable of selectively binding a Jak2 JH2 V617F. In addition, the invention provides the crystallographic information necessary to determine crystal structures of the Jak, particularly the Jak JH2, and more particularly a Jak2 JH2 V617F, in combination with the agents such as small molecules.

In a fifth aspect, the invention provides computer-assisted methods for selecting an agent or compound capable of binding to a Jak, particularly a Jak JH2, and more particularly a Jak2 JH2 V617F, comprising screening a plurality of agents or compounds to determine the ability of a test agent or compound to fit into a three-dimensional structure formed by the Jak, particularly the Jak JH2, and more particularly the Jak2 JH2 V617F, and selecting a test agent or compound from the plurality which is predicted to fit into the three-dimensional space.

In one embodiment of the invention, the three-dimensional structure is the Jak, particularly the Jak JH2, and more particularly a Jak2 JH2 V617F. The three dimensional structural coordinates are available publicly from, for instance, the RCSB Protein Data Bank managed by Rutgers, the State University of New Jersey, Center for Integrative Proteomics Research, 174 Frelinghuysen Rd., Piscataway, N.J. 08854-8076 and San Diego Computer Center (SDSC), and Skaggs School of Pharmacy and Pharmaceutical Sciences, University of California, San Diego (UCSD) 9500 Gilman Drive, La Jolla, Calif. 92093-0537. The crystal structures are provided in three files as 4FVP: Crystal structure of the Jak2 pseudokinase domain (apo form), 4FVQ: Crystal structure of the Jak2 pseudokinase domain (Mg-ATP-bound form), and 4FVR: Crystal structure of the Jak2 pseudokinase domain mutant V617F (Mg-ATP-bound form). In another embodiment, the computer-assisted methods are, for example, virtual ligand docking and screening techniques capable of designing and/or identifying a compound predicted to bind to a three-dimensional motif of the Jak, particularly the Jak JH2, and more particularly the Jak2 JH2 V617F. A three-dimensional motif of the Jak, particularly the Jak JH2, and more particularly the Jak2 JH2 V617F is provided herein. Agents and compounds may be designed and/or identified that are predicted to bind to three-dimensional motifs with a range of different affinities. In one embodiment, a compound is designed and/or identified that is predicted to bind to a three-dimensional motif with high affinity.

In an embodiment of the invention, binding of a compound to a three-dimensional motif of the a Jak, particularly the Jak JH2, and more particularly the Jak2 JH2 V617F is predicted to modulate an activity of the Jak, particularly the Jak JH2, and more particularly the Jak2 JH2 V617F. In one embodiment, modulating an activity of the Jak, particularly the Jak JH2, and more particularly the Jak2 JH2 V617F reduces or inhibits an activity of the Jak, particularly the Jak JH2, and more particularly the Jak2 JH2 V617F. Alternatively, modulating an activity of the Jak, particularly the Jak JH2, and more particularly the Jak2 JH2 V617F may increase or prolong an activity of the Jak, particularly the Jak JH2, and more particularly the Jak2 JH2 V617F.

In a specific embodiment of the method of the invention, agents such as ligands and small molecules capable of discriminating between the Jak and the Jak2 JH2 V617F are designed and/or identified by the computer assisted methods described herein. Compounds identified by the methods of the invention may be subsequently tested in in vitro assays as described herein determine their ability to selectively bind to the Jak2 JH2 V617F and modulate (i.e., inhibit or activate) an activity of the Jak, JH2 and/or Jak2 JH2 V617F.

In a sixth aspect, the invention provides agents, for example ligands and small molecules, capable of selectively binding to a Jak, a Jak JH2 or a Jak2 JH2 V617F. In particular, such agents, for example ligands and small molecules, may be capable of binding a Jak2 JH2 V617F selectively. That is, such agents, for example ligands and small molecules, may not appreciably bind to a Jak JH2 (WT). Similarly, the present invention provides agents, for example ligands and small molecules, identified by the methods described herein.

In a seventh aspect, the present invention provides methods for treating a myeloproliferative neoplasias (MPN), including, for instance, polycythemia vera, primary myelofibrosis, essential thrombocythemia, acute lymphoblastic leukemia, and acute myeloid leukemia by administering an agent that selectively inhibits a Jak, a Jak JH2 or a Jak2 JH2 V617F. The agent may be identified by the methods described herein. The agent may be, for instance, a small molecule.

Particular features and advantages of the invention will be apparent from the detailed description, the drawings, and the claims.

### BRIEF DESCRIPTION OF THE DRAWINGS

FIG. 1 depicts the Jak-Stat signaling pathway (from Yamaoka et al., Genome Biology 5:253 (2004)). Cytokine binding to its cognate receptor induces receptor dimerization (or re-arrangement of the receptor dimer) and trans-phosphorylation of associated Jaks. The activated Jaks phosphorylate the receptors on tyrosine residues, which become recruitment sites for the SH2 domains of Stats. Stats phosphorylated by Jaks dimerize and translocate to the nucleus to activate gene transcription.

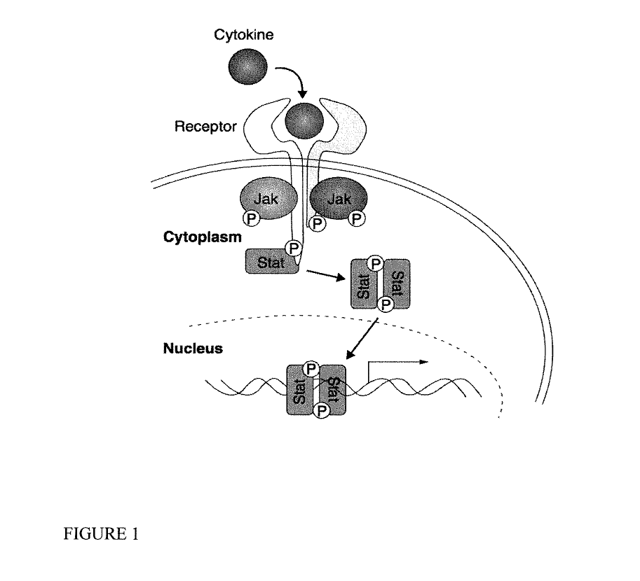

FIG. 2 provides a schematic diagram of human Jak2. All Jaks contain an N-terminal FERM domain, followed by an SH2-like domain, a pseudokinase domain (PKD/JH2), and a C-terminal tyrosine kinase domain (TKD/JH1). Human Jak2 is 1132 residues in length, and the domains are shown to linear scale. The location of well established sites of autophosphoryl-ation are indicated, with activating phosphorylation sites shown in green and negative regulatory sites shown in red. Also shown is the location of the most common activating mutation in MPNs, V617F in JH2.

FIG. 3 provides the crystal structure of Jak2 JH2. (a) Ribbon diagram of JH2-VF in complex with Mg-ATP. The MPN-causing mutation V617F is colored magenta (labeled ‘F617’). The N lobe is colored light gray and the C lobe is colored dark gray, save for the following segments: catalytic loop, orange; activation loop, green; ATP-binding loop, blue; αC (labeled) yellow. ATP is shown in stick representation, and the Mg²⁺ ion is shown as a purple sphere. The N- and C-termini are labeled (Nt, Ct). (b) Cα trace of a superposition between JH2-WT (white) and JH2-VF (cyan). The view is approx. 90° from the right of (a), with αC (labeled) in the foreground. L583, F594, and F595 are shown in stick representation, as is the MPN mutation V617F. A water molecule that intercalates in αC of JH2-WT (but not in JH2-VF) is shown as a red sphere.

FIG. 4 depicts source Q purification of JH2-WT(513). The protein elutes in two major peaks during development of the salt gradient. P1 is unphosphorylated JH2 and P2 is stoichiometrically phosphorylated on 5523.

FIG. 5 depicts ATP binding to JH2-WT. The decrease of the tryptophan emission signal at 340 nm (excitation wavelength at 285 nm) resulting from FRET upon mant-ATP binding to JH2 was measured as a function of mant-ATP concentration. The plotted data points are an average of two experiments.

FIG. 6 provides two possible models for the regulation of Jak2 by JH2. Jak2 (FERM, SH2, JH2, and JH1) is shown bound to a cytokine receptor (light gray). The plasma membrane is depicted by the brown rectangle. The different colors of JH1 reflect different levels of catalytic activity, from magenta (low) to dark green (medium) to bright green (high). In Model 1 (top), an equilibrium exists between state I, in which an autoinhibitory interaction between JH2 and JH1 maintains JH1 in a low activity state, and state II, in which a stimulatory interaction between JH2 and JH1 makes JH1 trans-phosphorylation competent (JH1 activity=medium). Trans-phosphorylation of Tyr1007/1008 in JH1 activates Jak2 (state III). Without cytokine present (upper path), state I is favored over state II. In the presence of cytokine (lower path), a rearrangement of the receptors favors formation of the JH2-JH1 stimulatory interaction (state II). MPN mutations such as V617F shift the equilibrium in favor of state II (vs. state I) in the absence of cytokine (upper path). In Model 2 (bottom), an equilibrium exists between state I, in which an inhibitory JH2 dimer sterically prevents JH1 from undergoing trans-phosphorylation, and state II, in which a stimulatory JH2 dimer facilitates JH1 trans-phosphorylation. Cytokine binding (lower path) rearranges the receptors, favoring formation of the stimulatory JH2 dimer (state II), which leads to trans-phosphorylation of Tyr1007/1008 in JH1 (state III). As in Model 1, MPN mutations such as V617F shift the equilibrium in favor of state II (vs. state I) in the absence of cytokine (upper path).

FIG. 7 demonstrates purification of JH2-JH1 (JH21) of Jak2. JH21 was expressed in baculovirus-infected Sf9 cells and purified by Ni-NTA chromatography, cleaved with TEV, and then purified by size-exclusion chromatography (Superdex 75). (a) Concentrated pool of Superdex 75 fractions. The yield after the Superdex column, from 1 L of Sf9 culture, is approximately 1.5 mg of protein. (b) OD280 trace of JH21 run on a Source Q column (after Superdex 75) without incubation with Mn²⁺-ATP (black trace) and after incubation with Mn²⁺-ATP (superimposed blue trace). Note the difference in ratio between peaks 1 and 4 before (black trace) and after (blue trace) autophosphorylation.

FIG. 8 demonstrates that K581A suppresses the constitutive activity of Jak2 V617F. (a) Full-length, HA-tagged Jak2, WT or indicated mutant, was transfected into Jak2-deficient γ2A cells, immunoprecipitated with anti-HA antibodies, and immunoblotted with phosphospecific Jak2 pY1007/1008 antibodies (top blot) or anti-HA antibodies (bottom blot, loading control for Jak2). NT=non-transfected. (b) Same as (a) but the cells were co-transfected with HA-Jak2 and HA-Stat1, and stimulated or not with IFNγ. Western blotting was performed with phosphospecific anti-Stat1 antibodies (top blot) or anti-HA antibodies (bottom blot, loading control for both Jak2 and Stat1).

FIG. 9 provides mapping of Jak2 MPN mutations onto the crystal structure of JH2. Mutated residues are shown in stick representation and colored magenta. Coloring of the ribbon is the same as in FIG. 3a. The view is similar to that in FIG. 3b.

FIG. 10 shows the phosphorylation state of additional MPN-causing Jak2 mutants. Full-length, HA-tagged Jak2, WT or indicated mutant, was transfected into Jak2-deficient γ2A cells, immunoprecipitated with anti-HA antibodies, and immunoblotted with the antibodies shown to the right of the blots. (a) and (b) represent two experiments with two different sets of Jak2 mutants. NT=non-transfected. The anti-HA blots are the loading controls.

FIG. 11 demonstrates identification of JAK2 JH2 catalytic activity in vitro. (a) In vitro kinase assay with purified JAK2 GST-JH2 with [³²P] γ-ATP in the absence or presence of divalent cations. (b) Time-course kinase assay with purified JAK2 GST-JH2 in the presence of [γ-³²P] ATP or unlabeled ATP. (c) Autoradiography of kinase assay (30 min) using purified JAK2 JH2 and JH1 domain and [γ-³²P] ATP, in the absence or presence of cations. Coomassie staining shows the protein levels of JH1 and JH2.

FIG. 12 demonstrates identification of phosphorylated residues in JAK2 JH2. (a) Chromatogram of JAK2 JH2 purification showing the peaks from anion-exchange chromatography. (b) Coomassie staining of a native-gel electrophoresis of JH2-Peak1 and JH2-Peak2 proteins. (c) Coomassie staining of a native-gel electrophoresis of purified JH2-Peak1 and JH2-Peak2 after kinase reaction. (d) MS-MS spectra of the phosphorylated residues in JAK2 JH2-Peak2 4 h kinase assay. Left: JH2-Peak2 is stoichiometrically phosphorylated at Ser523. Right: JH2-Peak2 is partially phosphorylated at Tyr570.

FIG. 13 provides an analysis of JAK2 JH2 autophosphorylation and ATP binding activity. (a) Time-course kinase assay of purified JH2-Peak1 and JH2-Peak2. (b) Time-course kinase assay of purified JH2 S523A and Y570F mutants compared to JH2-Peak2. (c) Fluorescence measurement of ATP binding assay of JAK2 JH2-Peak2. (d) K_{d} measurement of mant-ATP binding to JH2-Peak2. Graph, mean±s.d. of three independent experiments.

FIG. 14 provides an analysis of JAK2 signaling in mammalian cells. (a, c) Phosphorylation of JAK2WT and mutants thereof in JAK2-deficient γ 2A cells. HA-tagged JAK2 proteins were immunoprecipitated with anti-HA antibody and JAK2 phosphorylation is shown by Western blotting. Anti-HA Western blots show protein levels for each independent experiment. (b) Phosphorylation of JAK2 JH2 in γ 2A cells. (d, e) Phosphorylation of STAT1 in response to IFN-γ stimulation and phosphorylation of STATS in response to Epo stimulation in γ2A cells. (f) Effect of JAK2 K581A mutation on STAT1 transcription activation using IF γ γ-dependent GAS luciferase reporter. Graph, mean±s.d. of three independent experiments, P<0.05. (g) Effect of JAK2 K581A mutation on STATS transcription activation using SPI-Luc2 luciferase reporter. The basal JAK2 WT activity was set to 1 for all experiments, graph, mean±s.d. of six independent experiments are shown, P<0.05.

FIG. 15 demonstrates phosphorylation of different JAK2 MPN mutants. (a) Phosphorylation of JAK2WT and MPN mutants in JAK2-deficient γ 2A cells. (b) Phosphorylation of JAK2 JH2 in γ 2A cells.

FIG. 16 also demonstrates the crystal structure of Jak2 JH2. (a) Ribbon diagram of the structure of JH2. The N lobe of JH2 is colored light gray expect for the nucleotide-binding loop (blue) and αC (yellow). The C lobe is colored dark gray except for the catalytic loop (orange) and the activation loop (green). The α helices and β strands are labeled (shown semi-transparent), as are the N- and C-termini (N and C). ATP is shown in stick representation and colored cyan (carbon), red (oxygen), blue (nitrogen) or black (phosphorus), and the Mg²⁺ ion is colored purple. The side chain of Val617, the site of the pathogenic mutation V617F (in the β4-β5 loop), is shown in stick representation. (b) Mode of ATP binding in JH2. The viewing angle is approximately the same as in a. Select side chains are shown, with carbon atoms colored according to the residue's location, as in a (e.g., orange for catalytic loop). Superimposed is an electron density map (Fₒ-F_{c}, pink mesh, contoured at 3σ) computed without Mg-ATP in the model (but present during refinement). Hydrogen bonds and salt bridges are represented by black dashed lines, and Mg²⁺ coordination (≦2.1 Å) is represented by green dashed lines.

FIG. 17 shows cis- versus trans-autophosphorylation of Jak2 JH2. Autophosphorylation reactions were performed at three different JH2 concentrations (¼x, most dilute), and aliquots were taken at the indicated time points. Equal amounts of protein were loaded in each lane for SDS-PAGE and autoradiography. Top: the starting sample is unphosphorylated JH2, which becomes phosphorylated at Ser523 (Ungureanu, et al., Nat. Struct. Mol. Biol., 2011, 18: 971-976). Bottom: the starting sample is Ser523-phosphorylated JH2, which becomes phosphorylated at Tyr570.

FIG. 18 provides a comparison of JH2 V617F and wild-type structures. (a) Cα trace comparing JH2-VF to JH2-WT in the N lobe. The view is approximately 90° from that in FIG. 1a, with the same coloring of secondary-structure elements for JH2-WT (e.g., yellow for αC). JH2-VF is colored pink. The side chains of Phe594, Phe595 and Val or Phe617 are shown in stick representation. Mg-ATP is represented as in FIG. 1a. The N-terminus (residue 536) is labeled N. The catalytic loop (C loop), the activation loop (A loop), αC and other select segments are labeled. (b) The view is rotated by 90° from that in a, and the molecular surfaces of Phe594, Phe595 and Phe617 are included. (c) Results of molecular dynamic simulations for wild-type (WT), V617F and F595A V617F JH2. The secondary-structure assignment for each residue in the αC region (amino acids (a.a.) 587-602), as given by DSSP (Kabsch, et al., Biopolymers, 1983; 22: 2577-2637), is plotted top-to-bottom as a function of simulation time (20 μs total). Residues in α-helical conformation are colored blue, and residues in all other conformations (e.g., coil, turn, 3₁₀ helix, etc.) are colored yellow.

FIG. 19 demonstrates basal activation state of Jak2 mutants in mammalian cells. Lysates from transiently transfected (or not, ‘-’) γ2A cells were immunoprecipitated with anti-HA antibodies and immunoblotted with either anti-pJak2 (pY1007-1008) antibodies (top) or anti-HA antibodies (bottom; Jak2 expression control). These data are all from the same two blots.

### DETAILED DESCRIPTION OF THE INVENTION

Before the present assay methodology and treatment methodology are described, it is to be understood that this invention is not limited to particular assay methods, or test compounds and experimental conditions described, as such methods and compounds may vary. It is also to be understood that the terminology used herein is for the purpose of describing particular embodiments only, and is not intended to be limiting, since the scope of the present invention will be limited only the appended claims.

### DEFINITIONS

As used in this specification and the appended claims, the singular forms “a”, “an” and “the” include plural references unless the context clearly dictates otherwise. Thus for example, references to “the method” include one or more methods, and/or steps of the type described herein and/or which will become apparent to those persons skilled in the art upon reading this disclosure and so forth.

The term “amino acid” within the scope of the present invention and as used in its broadest sense, is meant to include the naturally occurring L alpha amino acids or residues. The commonly used one- and three-letter abbreviations for naturally occurring amino acids are used herein (Lehninger, Biochemistry, 2d ed., pp. 71-92, (Worth Publishers: New York, 1975). The term includes D-amino acids as well as chemically-modified amino acids such as amino acid analogs, naturally occurring amino acids that are not usually incorporated into proteins such as norleucine, and chemically-synthesized compounds having properties known in the art to be characteristic of an amino acid. For example, analogs or mimetics of phenylalanine or proline, which allow the same conformational arrangement of the peptide compounds as natural Phe or Pro, are included within the definition of amino acid. Such analogs and mimetics are referred to herein as “functional equivalents” of an amino acid. Other examples of amino acids are listed by Roberts and Vellaccio, The Peptides: Analysis, Synthesis, Biology, Eds. Gross and Meiehofer, Vol. 5, p. 341 (Academic Press, Inc.: N.Y. 1983). The term “amino acid” also has further, more detailed measuring as the latter pertains to the description of the invention, which usage and more detailed meaning is set forth in Paragraph 0080, infra.

The term “conservative” amino acid substitution as used herein to refer to amino acid substitutions that substitute functionally-equivalent amino acids. Conservative amino acid changes result in silent changes in the amino acid sequence of the resulting peptide. For example, one or more amino acids of a similar polarity act as functional equivalents and result in a silent alteration within the amino acid sequence of the peptide. The largest categories of conservative amino acid substitutions include: hydrophobic, neutral hydrophilic, polar, acidic/negatively charged, neutral/charged, basic/positively charged, aromatic, and residues that influence chain orientation. One of ordinary skill in the art is aware of the amino acid residues that are categorized within any one of the above categories and may, therefore, be conservatively substituted. In addition, “structurally-similar” amino acids can substitute conservatively for some of the specific amino acids. Groups of structurally similar amino acids include: Leu, and Val; Phe and Tyr; Lys and Arg; Gln and Asn; Asp and Glu; and Gly and Ala. In this regard, it is understood that amino acids are substituted on the basis of side-chain bulk, charge, and/or hydrophobicity. Amino acid residues are classified into four major groups: acidic, basic, neutral/non-polar, and neutral/polar.

An acidic residue has a negative charge due to loss of an H ion at physiological pH and is attracted by aqueous solution so as to seek the surface positions in the conformation of a peptide in which it is contained when the peptide is in aqueous solution.

A basic residue has a positive charge due to association with an H ion at physiological pH and is attracted by aqueous solution so as to seek the surface positions in the conformation of a peptide in which it is contained when the peptide is in aqueous medium at physiological pH.

A neutral/non-polar residue is not charged at physiological pH and is repelled by aqueous solution so as to seek the inner positions in the conformation of a peptide in which it is contained when the peptide is in aqueous medium. These residues are also designated “hydrophobic residues.”

A neutral/polar residue is not charged at physiological pH, but the residue is attracted by aqueous solution so as to seek the outer positions in the conformation of a peptide in which it is contained when the peptide is in aqueous medium.

“Amino acid” residues can be further classified as cyclic or non-cyclic, and aromatic or non-aromatic with respect to their side-chain groups, these designations being commonplace to the skilled artisan.

Peptides of the invention can be synthesized by standard solid-phase synthesis techniques. Such peptides are not limited to amino acids encoded by genes for substitutions involving the amino acids. Commonly encountered amino acids that are not encoded by the genetic code include, for example, those described in WO 90/01940, as well as, for example, 2-amino adipic acid (Aad) for Glu and Asp; 2-aminopimelic acid (Apm) for Glu and Asp; 2-aminobutyric (Abu) acid for Met, Leu, and other aliphatic amino acids; 2-aminoheptanoic acid (Ahe) for Met, Leu, and other aliphatic amino acids; 2-aminoisobutyric acid (Aib) for Gly; cyclohexylalanine (Cha) for Val, Leu and Ile; homoarginine (Har) for Arg and Lys; 2,3-diaminopropionic acid (Dpr) for Lys, Arg, and His; N-ethylglycine (EtGly) for Gly, Pro, and Ala; N-ethylglycine (EtGly) for Gly, Pro, and Ala; N-ethylasparagine (EtAsn) for Asn, and Gin; hydroxylysine (Hyl) for Lys; allohydroxylysine (AHyl) for Lys; 3-(and 4-)hydroxyproline (3Hyp, 4Hyp) for Pro, Ser, and Thr; allo-isoleucine (Alle) for lie, Leu, and Val; .rho.-amidinophenylalanine for Ala; N-methylglycine (MeGly, sarcosine) for Gly, Pro, and Ala; N-methylisoleucine (Melle) for Ile; norvaline (Nva) for Met and other aliphatic amino acids; norleucine (Nle) for Met and other aliphatic amino acids; ornithine (Orn) for Lys, Arg and His; citruline (Cit) and methionine sulfoxide (MSO) for Thr, Asn, and Gin; and N-methylphenylalanine (MePhe), trimethylphenylalanine, halo-(F—, Cl—, Br—, or I) phenylalanine, or trifluorylphenylalanine for Phe.

As used herein, the term “modulator” refers to a compound capable of modulating, altering, or changing an activity of a molecule. In the context of the present invention, a modulator may be used to alter an activity of a Jak, particularly a Jak JH2, and more particularly a Jak2 V617F or a functional fragment thereof. In a particular embodiment, a modulator may alter an activity associated with a kinase domain of the Jak, JH2, and more particularly the Jak2 V617F or a fragment thereof. The term “modulator,” “modulatory compound,” or “modulatory agent” encompasses a compound/agent capable of decreasing, inhibiting, and/or reducing an activity of a molecule (i.e., an inhibitor) or increasing, enhancing, and/or prolonging an activity of a molecule (i.e., an activator).

An inhibitor of the Jak, particularly the Jak JH2, and more particularly the Jak2 V617F, for example, is a compound/agent capable of decreasing, inhibiting, and/or reducing an activity of the Jak, particularly the Jak JH2, and more particularly the Jak2 V617F. It is to be understood that a compound/agent capable of inhibiting the Jak, particularly the Jak JH2, and more particularly the Jak2 V617F may be specific for an activity of the Jak, particularly the Jak JH2, and more particularly the Jak2 V617F.

An activator of the Jak, particularly the Jak JH2, and more particularly the Jak2 V617F, for example, is a compound/agent capable of increasing, enhancing, and/or prolonging an activity of the Jak, particularly the Jak JH2, and more particularly the Jak2 V617F. It is to be understood that a compound/agent capable of “activating” or “prolonging the activated state” of the Jak, particularly the Jak JH2, and more particularly the Jak2 V617F may be specific for an activity of the Jak, particularly the Jak JH2, and more particularly the Jak2 V617F.

As used herein, a “three-dimensional motif” refers to a spatial conformation formed by an association or arrangement of different amino acid residues and/or regions of a molecule. The nature of such associations and arrangements is discussed in detail below.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated by reference to disclose and describe the methods and/or materials in connection with which the publications are cited.

A structural understanding of the autoregulation of Jaks by the pseudokinase domain is one of the important unsolved problems in the field of tyrosine kinase signaling. This mechanistic problem is made even more compelling by the recent finding that JH2 is an active protein kinase. A crystal structure of JH2 will provide, along with supporting biochemical data, the molecular basis for the catalytic activity of JH2, and a crystal structure of JH2-JH1 will provide the mechanism by which JH2 autoinhibits JH1. Moreover, the crystal structures will allow rationalization of mutations in JH2 (e.g., V617F) that cause MPNs, and should facilitate the development of novel inhibitors to combat these diseases.

Although there has been great interest over the years in obtaining a crystal structure of a Jak protein that includes the pseudokinase domain (JH2), to understand the autoinhibitory mechanism mediated by JH2, the main hurdle has been the inability to express and purify sufficient quantities of soluble protein for structural studies. The present invention overcomes this hurdle, and provides expressing Jak2 JH2 and JH2-JH1 in soluble form in baculovirus-infected Sf9 insect cells. In addition, the present invention provides a triple mutant of JH2 that dramatically improves solubility and has yielded crystals of JH2. Thus, it is now possible to provide crystallographic studies of the kinase domains of Jak2 to elucidate the autoregulatory mechanism(s) mediated by JH2.

The pseudokinase domain inhibits Jak2 kinase activity (Saharinen, et al., Mol. Cell. Biol. 2000; 20: 3387-3395). Various baculoviruses (encoding JH2 and JH2-JH1, wild-type and mutants) and the expression of the Jak2 proteins is performed in Sf9 cells. The crystallization and structure of protein tyrosine kinases have been determined for numerous structures (in various phosphorylation states and in complex with small-molecule inhibitors or with other signaling proteins) of the tyrosine kinase domains of the insulin and insulin-like growth factor-1 receptors (Hubbard, et al., Nature 1994; 372: 746-754; Hubbard, EMBO J. 1997; 16: 5572-5581; Depetris, et al., Mol. Cell. 2005; 20: 325-333; Hu, et al., Mol. Cell. 2003; 12: 1379-1389; Li, et al., Structure 2005; 13: 1643-1651; Li, et al., J. Biol. Chem. 2003; 278: 26007-26014; Parang, et al., Nat. Struct. Biol. 2001; 8: 37-41; Favelyukis, et al., Nat. Struct. Biol. 2001; 8: 1058-1063; Wu, et al., EMBO J. 2008; 27: 1985-1994; Wu, et al., Nat. Struct. Mol. Biol. 2008; 15: 251-258), fibroblast growth factor receptor (Mohammadi, et al., Cell 1996; 86: 577-587; Mohammadi, et al., Science 1997; 276: 955-960; Mohammadi, et al., EMBO J. 1998; 17: 5896-5904), and muscle-specific kinase (Till, et al., Structure 2002; 10: 1187-1196).

A key to crystallizing protein kinases is to purify a single phosphorylation state of the enzyme and to capture a single conformational state. Protein kinases are bi-lobed enzymes (N and C lobes) in which the phosphate donor, ATP, binds in the cleft between the two lobes, and the serine/threonine- or tyrosine-containing substrate binds in the active site in the C lobe (Taylor, et al., Structure 1994; 2: 345-355; Hubbard, et al., Annu. Rev. Biochem. 2000; 69: 373-398). There is considerable conformational plasticity in protein kinases, especially in the relative orientation of the N and C lobes (Huse, et al., Cell 2002; 109: 275-282). For protein kinases in an active state, particularly those activated by phosphorylation of the activation loop (like JH1 of Jak2), non-hydrolyzable ATP analogs or small-molecule inhibitors are often required to stabilize the relative position of the two lobes.

### Structural and Biochemical Characterization of the Pseudokinase Domain (JH2) of Jak2

Jak2 JH2 (human, residues 536-812), both wild-type (JH2-WT) and the activating mutant V617F (JH2-VF), have been expressed in soluble form in baculovirus-infected Sf9 cells (constructs include an N-terminal 6× His-tag). JH2 is purified on Ni-NTA and anion-exchange columns (Source Q). Because this construct lacks the autophosphorylation site S523 (SH2-JH2 linker), and Y570 within JH2 is not phosphorylated in the absence of S523 phosphorylation, the protein elutes from a Source Q column in a single peak. To increase the solubility of the proteins, hydrophobic residues on the surface of JH2 were identified (based on a model for JH2), for which substitution with alanine or a hydrophilic residue might increase solubility. Several putative surface-exposed hydrophobic residues were identified and three substitutions were made: W659A (not conserved in the other Jaks/Tyk2), W777A (conserved in Jak3 but not Jak1 or Tyk2) and F794H (glutamine in the other Jaks/Tyk2). In fact, in mammalian cells, the expression and phosphorylation levels of full-length Jak2 bearing the three JH2 mutations were indistinguishable from wild-type Jak2 (data not shown). These three mutations were introduced into JH2-WT and -VF, and the resultant proteins were expressed at higher levels, were much better behaved during purification and yielded crystals readily.

The following crystal structures were determined:

1) JH2-WT without nucleotide, 2.05 Å resolution (R_{free}=22%)
2) JH2-WT with Mg-ATP, 2.1 Å resolution (R_{free}=23%)
3) JH2-VF with Mg-ATP, 2.5 Å resolution (R_{free}=26%)

The initial structure of JH2-WT (without nucleotide) was determined by molecular replacement, using the C lobe of the epidermal growth factor receptor kinase as a search model.

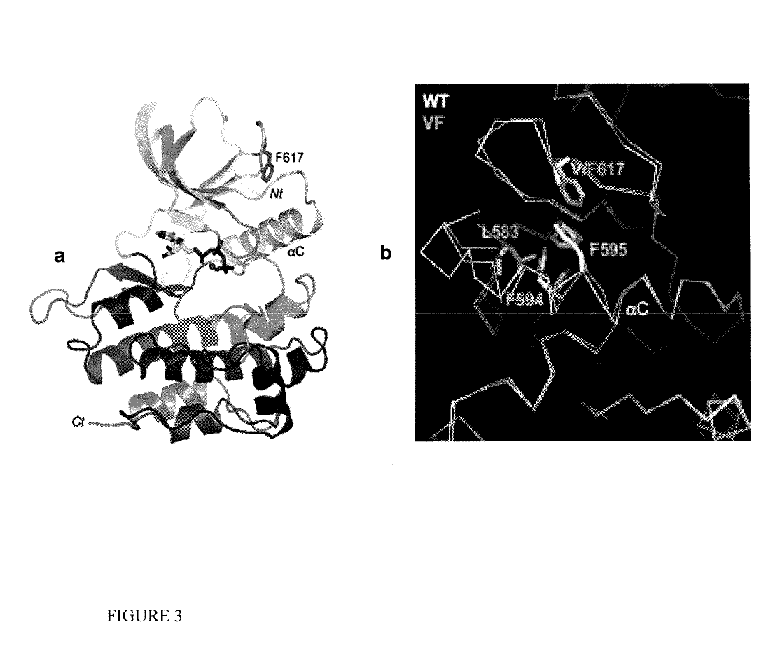

Jak2 JH2 adopts a canonical protein-kinase fold, with an N lobe comprising a five-stranded β sheet and one a helix (αC), and a C lobe that is mainly a helical (FIG. 3a). Some of the salient features of the JH2-WT and -VF structures are:
1) The activation loop is ordered throughout and terminates in an a helix, which is novel for a kinase activation loop.
2) The DFG sequence at the beginning of the activation loop in canonical protein kinases is replaced by DPG in JH2 of Jaks. The aspartic acid (D699) in this motif, rather than coordinating a Mg²⁺ ion, as in canonical protein kinases, is salt-bridged to conserved K581 in β-strand 3 (β3). Normally, this lysine is salt-bridged to a conserved glutamic acid in αC, which is absent in JH2 of Jaks (alanine instead). The aspartic acid in the DPG motif clearly substitutes for the glutamic acid missing in αC.
3) The Mg²⁺ ion is coordinated by all three phosphate groups of ATP and by N678 in the catalytic loop. This represents a novel Mg²⁺ coordination scheme for a protein kinase.
4) In the JH2-WT structure, there is a kink (disruption of backbone hydrogen-bonding) in the middle of αC, at F595, just below V617, the site of the MPN mutation V617F (FIG. 3b). A water molecule is intercalated in the helix.
5) The structure of JH2-VF (crystallized in the same lattice as that of JH2-WT and also in a second lattice) is very similar to that of JH2-WT (rmsd in Cα positions of 0.44 Å), except for the regions proximal to the mutation.
6) In JH2-VF, the mutation of V617 to phenylalanine causes αC to revert to a continuous a helix (uninterrupted backbone hydrogen-bonding). This structural alteration in αC is likely to be a key feature of the mechanism by which V617F causes constitutive activation of Jak2. Another structural effect of the mutation is to alter the positioning of F594 in αC, forcing it closer to the JH2 active site (FIG. 3b). This may inhibit the catalytic activity of JH2; in full-length Jak2 bearing V617F, 5523 autophosphorylation is abrogated (Ungureanu, et al., Nat. Struct. Mol. Biol. AOP, Aug. 14 (2011)).

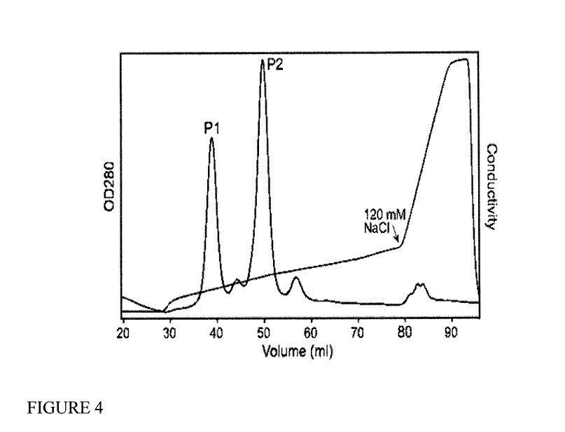

A longer (on the N-terminus) form of Jak2 JH2, residues 513-812, which includes the autophosphorylation site S523 in the SH2-JH2 linker was expressed and purified. On an anion-exchange column (Source Q), the protein elutes in two major peaks (FIG. 4), which were confirmed by mass spectrometry represent the unphosphorylated and S523-phosphorylated species of JH2.

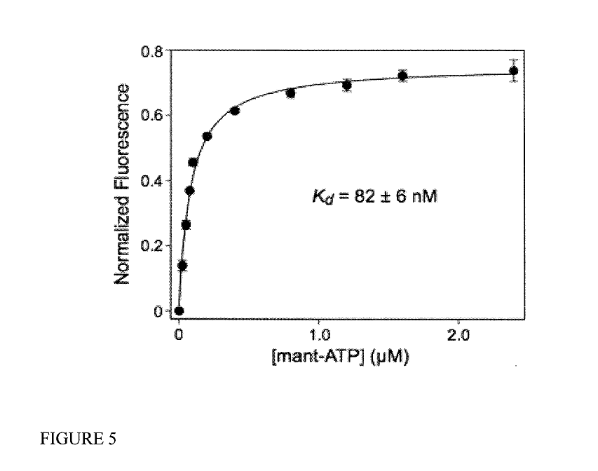

As a first step in characterizing the catalytic properties of Jak2 JH2, dissociation constants (K_{d} values) for ATP binding to JH2-WT and -VF were obtained using the fluorescent ATP analog mant-ATP. These measurements yielded a K_{d} of 82 nM for JH2-WT (FIG. 5) and a very similar value (84 nM) for JH2-VF. The ATP binding affinity is surprisingly high. As a comparison, the same measurement was performed with the insulin receptor kinase (phosphorylated, active form), a prototypical tyrosine kinase, and obtained a K_{d} of 14 μM, nearly ˜30-fold higher.

An interaction between JH2 and JH1 that maintains JH1 in a low-activity (basal) state was postulated a decade ago, and a crystal structure characterizing this interaction has been much anticipated ever since. A structural model was proposed in 2001 by Lindauer et al. (Lindauer, et al., Protein Eng. 2001; 14: 27-37), which was based on a crystallographic dimer of the fibroblast growth factor receptor kinase, a structure we solved back in 1996 (Mohammadi, et al., Cell 1996; 86: 577-587). We expect a structure of unphosphorylated JH2-JH1 to reveal the true molecular basis for this crucial autoinhibitory interaction which we be verified through mutagenesis studies. In addition, a JH2-JH1 structure should provide a wealth of structural data to understand the basis for the constitutive activity of V617F and of other MPN-causing JH2 mutations. A crystal structure of phosphorylated, activated JH2-JH1 will reveal the molecular basis for the role of JH2 in maintaining the active state of JH1.

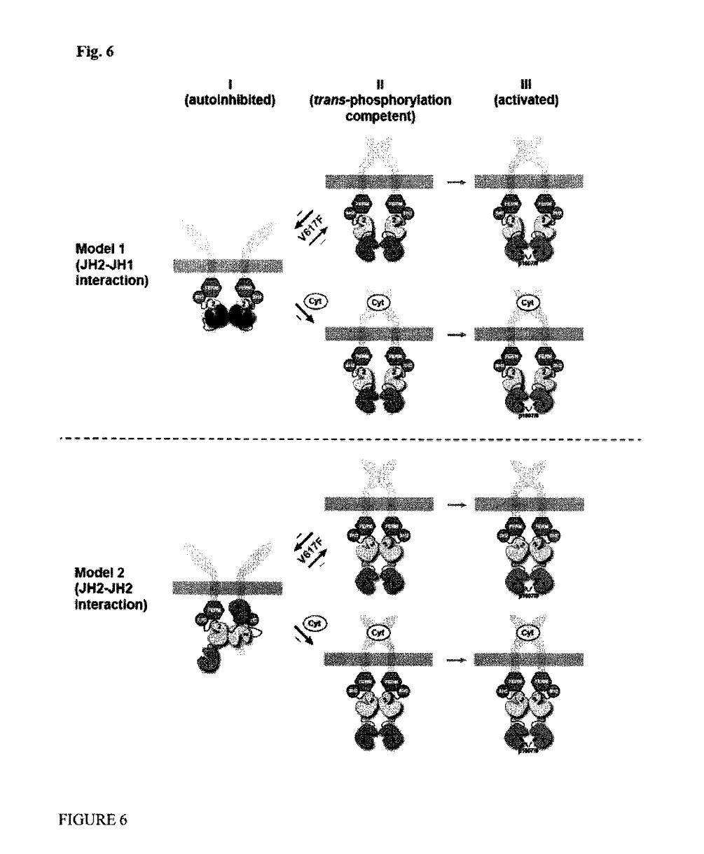

A model that may account for the dual negative and positive roles of JH2 in the regulation of JH1 kinase activity is depicted in FIG. 6, in which JH2 forms an autoinhibitory interaction with JH1 when the activation loop of JH1 is unphosphorylated (state I [autoinhibited]), and forms a stimulatory (stabilizing) interaction with JH1 after trans-phosphorylation of the JH1 activation loop (state IV [activated]). The constitutive activity of V617F (and other gain-of-function mutations) in JH2 is thought to derive from a destabilized JH2-JH1 autoinhibitory interaction, which shifts the conformational equilibrium from the autoinhibited state (I) to the autophosphorylation-permissive state (II), thereby facilitating cytokine-independent autophosphorylation of the JH1 activation loop and Jak activation (states III and IV).

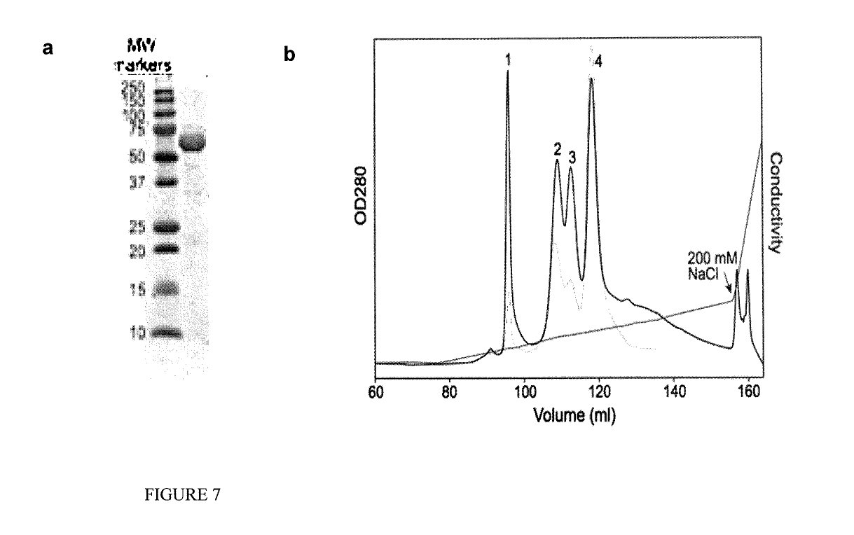

Jak2 JH2-JH1 (human, residues 536-1132), both wild-type and V617F (JH21-WT and -VF, respectively) were expressed in soluble form in baculovirus-infected Sf9 cells (FIG. 7). On a size-exclusion column, JH21 elutes at a position consistent with a monomeric protein (69.3 kDa calculated, data not shown). When run on a anion-exchange column, JH21 elutes in four peaks during development of the salt gradient (FIG. 7b, black trace). If the protein is first incubated with Mn²⁺-ATP, then run anion exchange, the same four peaks are present (FIG. 7b, blue trace), but there is a shift in the amount of JH21 to the later peaks. This behavior is indicative of an active protein kinase that is differentially phosphorylated (the higher the phosphorylation state, the later the elution position).

### Functional Mutagenesis of Jak2

From recent crystallographic and in vitro biochemical studies of Jak2 JH2, and from previously reported studies of Jaks, it is possible to formulate hypotheses regarding the mechanisms by which JH2 regulates Jak2 activity (see FIG. 6 for general model) and how mutations in JH2 lead to hyperactivity of Jak2 (JH1) and ultimately to disease (MPNs).

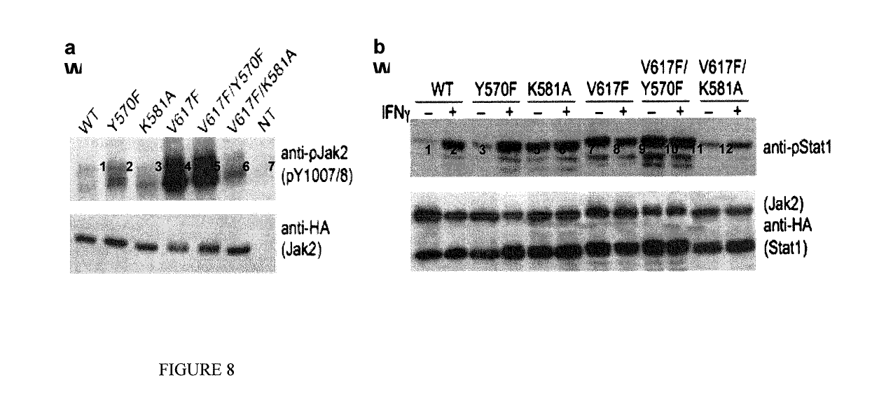

JH2 is an active protein kinase that phosphorylates two negative regulatory sites in Jak2, S523 and Y570. Mutation of either of these sites, or mutation of K581, the kinase-conserved lysine in β3 of JH2, leads to an increase in the phosphorylation state of Jak2 when transfected into Jak2-deficient γ2A cells, but not to the same level as the MPN-mutation V617F (FIG. 8a, compare lanes 1-4). FIG. 8a shows that K581A suppresses the constitutive activity of V617F (lane 6 vs. 4), whereas Y570F, mutation of one of the negative regulatory sites, did not suppress V617F (lane 5 vs. 4). K581A suppresses Stat1 phosphorylation by V617F (FIG. 8b, lanes 11-12 vs. lanes 7-8). This indicates that either the catalytic activity of JH2 (which should be abrogated by K581A) is important for the constitutive activation of V617F or that K581A destabilizes JH2 structurally (perhaps because it no longer binds ATP productively). A destabilized JH2 could lead to lower Jak2 (JH1) activity if the putative activating/stabilizing interaction between JH2 and JH1 is compromised (FIG. 6).

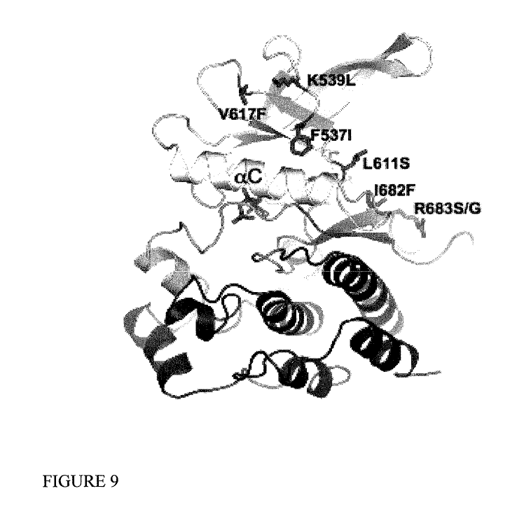

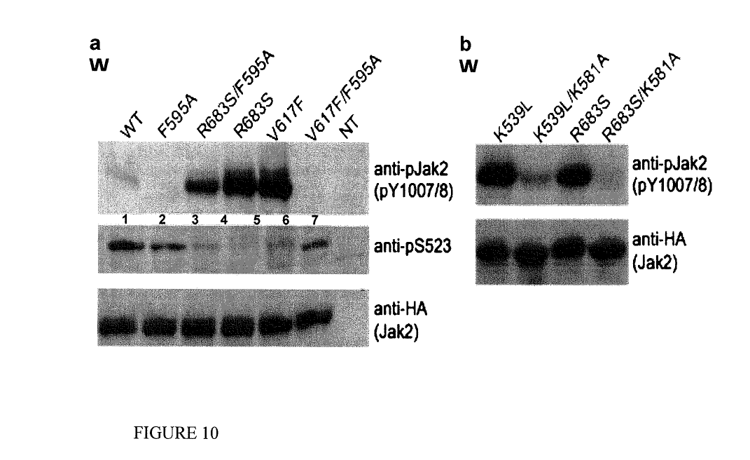

Other MPN-causing mutations in Jak2 JH2 have been examined (FIG. 9). Two such mutations are R683S (exon 16) in β7 and K539L (exon 12) at the beginning of the domain. When transfected into γ2A cells, the phosphorylation state of these two mutants is elevated, like for V617F (exon 14) (FIG. 10a, lane 4 vs. lanes 5 and 1 in top blot), and similar to V617F, these activating mutations are suppressed by the addition of K581A (FIG. 10b).

F595A suppresses V617F. This is now easily understood from the JH2 structures: F595 in αC sits just below V617 (FIG. 3b), and the larger phenylalanine in the mutant V617F sterically impinges on F595, resulting in a structural perturbation in αC. This perturbation would not occur with the smaller alanine at position 595, and hence V617F activity is suppressed. (FIG. 10a, lane 5 vs. 6 in top blot) F595A does not suppress the MPN mutation R683S to the same extent (FIG. 10a, lane 4 vs. 3 in top blot). Interestingly, S523 phosphorylation, which is catalyzed by JH2, decreases in both V617F and R683S (FIG. 10a, lanes 5 and 4 vs. 1 in middle blot) and can be restored in V617F but not in R683S by introducing F595A (FIG. 10a, lane 6 vs. 5 vs. 3 in middle blot). These data indicate that both V617F and R683S compromise JH2 catalytic activity, and suggest the possibility that hyperactivity of MPN-causing mutations in Jak2 results from loss of kinase activity as well as from loss of the putative autoinhibitory interaction between JH2 and JH1.

In view of the above, the present discovery also presents evidence that may be applied to the identification of small molecule modulators (e.g. inhibitors) that bind differentially to the Jak, particularly the Jak JH2, and more particularly the Jak2 V617F. Inasmuch as different disorders/diseases are associated with altered responses/regulation of one of these, there is a critical need to identify compounds capable of specifically modulating the Jak, particularly the Jak JH2, and more particularly the Jak2 V617F.

### General Methods

In one embodiment of the invention, the three-dimensional structural information of the Jak, particularly the Jak JH2, and more particularly the Jak2 V617F is used as a target in a virtual ligand screening procedure that seeks to identify, via computer docking methods, those candidate agents and compounds in a library that are capable of binding to the target site with high affinity. In another embodiment, the structural information of the Jak, particularly the Jak JH2, and more particularly the Jak2 V617F is used to design compounds predicted to bind to the Jak, particularly the Jak JH2, and more particularly the Jak2 V617F, and such compounds are tested for high affinity binding.

Compounds derived or obtained from either approach scoring the highest in the docking procedure are then tested in cell-based and cell-free assays (described below) to determine their efficacy in inhibiting the Jak, particularly the Jak JH2, and more particularly the Jak2 V617F. In one embodiment of the invention, an agent or compound identified by the instant methods blocks the Jak, particularly the Jak JH2, and more particularly the Jak2 V617F as a Jak antagonist.

As used herein, the term “modified peptide” may be used to refer to a peptide that is capable of binding to a protein and modulating its activity (e.g., a tyrosine kinase domain of a cell surface receptor). Modified peptides may possess features that, for example, modulate (increase or decrease) binding, alter the half-life of the peptide, decrease renal clearance, or improve absorption.

As used herein, the term “peptide” is used in its broadest sense to refer to compounds containing amino acid equivalents or other non-amino groups, while still retaining the desired functional activity of a peptide. Peptide equivalents can differ from conventional peptides by the replacement of one or more amino acids with related organic acids (such as PABA), amino acids or the like or the substitution or modification of side chains or functional groups.

It is to be understood that limited modifications can be made to a peptide without destroying its biological function. Thus, modified forms of peptides identified using a method of the invention are encompassed herein, as long as they retain an activity of the peptide. Modifications can include, for example, additions, deletions, or substitutions of amino acids residues, substitutions with compounds that mimic amino acid structure or functions, as well as the addition of chemical moieties such as amino or acetyl groups. The modifications can be deliberate or accidental, and can be modifications of the composition or the structure.

Selected compounds exhibiting desirable characteristics are designated lead compounds, and further tested in animal models to measure their efficacy.

### Virtual Ligand Screening Via Flexible Docking Technology

Knowledge pertaining to a specific receptor structure can be used in conjunction with current docking and screening methodologies to select small sets of likely lead candidate ligands from large libraries of agents and compounds. Such methods are described, for example, in Abagyan and Totrov (2001) Current Opinion Chemical Biology 5:375-382, herein specifically incorporated by reference in its entirety.

Virtual ligand screening (VLS) based on high-throughput flexible docking is useful for designing and identifying compounds capable of binding to a specific receptor structure. VLS can be used to virtually sample a large number of chemical molecules without synthesizing and experimentally testing each one. Generally, the methods start with receptor modeling which uses a selected receptor structure derived by conventional means, e.g., X-ray crystallography, NMR, homology modeling. A set of compounds and/or molecular fragments are then docked into the selected binding site using any one of the existing docking programs, such as for example, MCDOCK (Liu et al. J. Comput. Aided Mol. Des. 1999; 13:435-451), SEED (Majeux et al. Proteins 1999; 37:88-105; DARWIN (Taylor et al. Proteins 2000; 41:173-191; MM (David et al. J. Comput. Aided Mol. Des. 2001; 15:157-171. Compounds are scored as ligands, and a list of candidate compounds predicted to possess the highest binding affinities are generated for further in vitro and in vivo testing and/or chemical modification.

In one approach of VLS, molecules are “built” into a selected binding pocket prior to chemical generation. A large number of programs are designed to “grow” ligands atom-by-atom [see, for example, GENSTAR (Pearlman et al. J. Comput. Chem. 1993; 14:1184), LEGEND (Nishibata et al. J. Med. Chem. 1993; 36:2921-2928), MCDNLG (Rotstein et al. J. Comput-Aided Mol. Des. 1993; 7:23-43), CONCEPTS (Gehlhaar et al. J. Med. Chem. 1995; 38:466-472) or fragment-by-fragment [see, for example, GROUPBUILD (Rotsein et al. J. Med. Chem. 1993; 36:1700-1710), SPROUT (Gillet et al. J. Comput. Aided Mol. Des. 1993; 7:127-153), LUDI (Bohm, J. Comput. Aided Mol. Des. 1992; 6:61-78), BUILDER (Roe J. Comput. Aided Mol. Des. 1995; 9:269-282), and SMOG (DeWitte et al. J. Am. Chem. Soc. 1996; 118:11733-11744).

Methods for scoring ligands for a particular receptor are known which allow discrimination between the small number of molecules able to bind the receptor structure and the large number of non-binders. See, for example, Agagyan et al. 2001 supra, for a report on the growing number of successful ligands identified via virtual ligand docking and screening methodologies.

For example, Nishibata et al. J. Med. Chem. 1993; 36:2921-2928, describe the ability of a structure construction program to generate inhibitory molecules based on the three-dimensional structure of the active site of a molecule, dihydrofolate reductase. The program was able to predict molecules having a similar structure to four known inhibitors of the enzyme, providing strong support that new lead compounds can be obtained with knowledge pertaining to the three dimensional structure of the target. Similarly, Gillet et al. J. Computer Aided Mol. Design. 1993; 7:127-153 describe structure generation through artificial intelligence techniques based on steric constrains (SPROUT).

The invention provides methods for identifying agents and compounds (e.g., candidate compounds or test compounds) that bind with high affinity to the Jak, particularly the Jak JH2, and more particularly the Jak2 V617F. Agents and compounds identified by the screening method of the invention are useful as candidate anti-neoplasia agents, and/or in any condition which could be ameliorated by inhibition of the Jak, particularly the Jak JH2, and more particularly the Jak2 V617F. Such conditions include, but are not limited to polycythemia vera, primary myelofibrosis, essential thrombocythemia, acute lymphoblastic leukemia, and acute myeloid leukemia.

### Agents Identified by the Screening Methods of the Invention

The invention provides methods for identifying agents (e.g., candidate compounds or test compounds) that bind with high affinity to the Jak, particularly the Jak JH2, and more particularly the Jak2 V617F. Agents identified by the screening methods of the invention may be used as candidate therapeutics for neoplasia disorders.

Examples of agents, candidate compounds or test compounds include, but are not limited to, nucleic acids (e.g., DNA and RNA), carbohydrates, lipids, proteins, peptides, peptidomimetics, small molecules and other drugs. Agents can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145; U.S. Pat. No. 5,738,996; and U.S. Pat. No. 5,807,683, each of which is incorporated herein in its entirety by reference).

Examples of methods for the synthesis of molecular libraries can be found in the art, for example in: DeWitt et al. Proc. Natl. Acad. Sci. USA 1993; 90:6909; Erb et al. Proc. Natl. Acad. Sci. USA 1994; 91:11422; Zuckermann et al. J. Med. Chem. 1994; 37:2678; Cho et al. Science 1993; 261:1303; Carrell et al. Angew. Chem. Int. Ed. Engl. 1994; 33:2059; Carell et al. Angew. Chem. Int. Ed. Engl. 1994; 33:2061; and Gallop et al. J. Med. Chem. 1994; 37:1233, each of which is incorporated herein in its entirety by reference.

Libraries of compounds may be presented, e.g., presented in solution (e.g., Houghten, Bio/Techniques 1992; 13:412-421), or on beads (Lam, Nature 1991; 354:82-84), chips (Fodor Nature 1993; 364:555-556), bacteria (U.S. Pat. No. 5,223,409), spores (U.S. Pat. Nos. 5,571,698; 5,403,484; and 5,223,409), plasmids (Cull et al. Proc. Natl. Acad. Sci. USA 1992; 89:1865-1869) or phage (Scott and Smith Science 1990; 249:386-390; Devlin, Science 1990; 249:404-406; Cwirla et al. Proc. Natl. Acad. Sci. USA 1990; 87:6378-6382; and Felici, J. Mol. Biol. 1991; 222:301-310), each of which is incorporated herein in its entirety by reference.

### Screening Assays

Small molecules identified through the above described virtual ligand docking and screening methodologies may be further tested in in vitro and in vivo assays. In one embodiment, agents and compounds that interact with (i.e., bind to) the Jak, particularly the Jak JH2, and more particularly the Jak2 V617F, or a functional fragment thereof, are identified and/or confirmed in a cell-free assay system. In accordance with this embodiment, a native or recombinant Jak, particularly Jak JH2, and more particularly Jak2 V617F or fragment thereof is contacted with a candidate compound or a control compound and the ability of the candidate compound to interact with the Jak, particularly the Jak JH2, and more particularly the Jak2 V617F is determined. If desired, this assay may be used to screen a plurality (e.g. a library) of candidate compounds. In one embodiment, the Jak, particularly the Jak JH2, and more particularly the Jak2 V617F or fragment thereof is first immobilized by contacting it with, for example, an immobilized antibody which specifically recognizes and binds to it, or by contacting a purified preparation of the Jak, particularly the Jak JH2, and more particularly the Jak2 V617F or fragment thereof, with a surface designed to bind proteins. The Jak, particularly the Jak JH2, and more particularly the Jak2 V617F or fragment thereof may be partially or completely purified (e.g., partially or completely free of other polypeptides) or part of a cell lysate. Further, the Jak, particularly the Jak JH2, and more particularly the Jak2 V617F or fragment thereof may be a fusion protein comprising the Jak, particularly the Jak JH2, and more particularly the Jak2 V617F or fragment thereof or a biologically active portion thereof, and a domain such as glutathionine-S-transferase. Alternatively, Jak, particularly the Jak JH2, and more particularly the Jak2 V617F or fragment thereof can be biotinylated using techniques well known to those of skill in the art (e.g., biotinylation kit, Pierce Chemicals; Rockford, Ill.). The ability of a candidate compound to interact with the Jak, particularly the Jak JH2, and more particularly the Jak2 V617F or fragment thereof can be determined by methods known to those of skill in the art.

### Cell Based Assays

In another embodiment, agents and compounds that interact with (i.e., bind to) the Jak, particularly the Jak JH2, and more particularly the Jak2 V617F or fragment thereof are tested in a cell-based assay system. In accordance with this embodiment, cells expressing Jak, particularly the Jak JH2, and more particularly the Jak2 V617F or fragment thereof, are contacted with a candidate compound or a control compound and the ability of the candidate compound to interact with the Jak, particularly the Jak JH2, and more particularly the Jak2 V617F or fragment thereof is determined. If desired, this assay may be used to screen a plurality (e.g. a library) of candidate compounds. A cell, for example, can be of prokaryotic origin (e.g., E. coli) or eukaryotic origin (e.g., yeast or mammalian). Further, the cells can express the Jak, particularly the Jak JH2, and more particularly the Jak2 V617F or fragment thereof endogenously or be genetically engineered to express the same. In certain instances, the Jak, particularly the Jak JH2, and more particularly the Jak2 V617F or fragment thereof is labeled, for example with a radioactive label (such as ³²P, ³⁵S or ¹²⁵I) or a fluorescent label (such as fluorescein isothiocyanate, rhodamine, phycoerythrin, phycocyanin, allophycocyanin, o-phthaldehyde or fluorescamine) to enable detection of an interaction between the IGF1 receptor and a candidate compound. The ability of the candidate compound to bind to the Jak, particularly the Jak JH2, and more particularly the Jak2 V617F or fragment thereof can be determined by methods known to those of skill in the art. For example, the interaction between a candidate compound and the Jak, particularly the Jak JH2, and more particularly the Jak2 V617F or fragment thereof can be determined by flow cytometry, a scintillation assay, immunoprecipitation or western blot analysis.

In general, Jak, particularly the Jak JH2, and more particularly the Jak2 V617F or fragment thereof expressing cells are treated with modulators (e.g., inhibitors) of Jak, particularly the Jak JH2, and more particularly the Jak2 V617F or fragment thereof activity and cell lysates are generated from these treated cells. Jak, particularly the Jak JH2, and more particularly the Jak2 V617F or fragment thereof are isolated from the lysate by immunoprecipitation and analyzed for phosphotyrosine content.

Chinese Hamster Ovary (CHO) cells overexpressing wild type Jak, particularly the Jak JH2, and more particularly the Jak2 V617F or fragment thereof are an exemplary cellular model in which to perform such assays. Such cells may be maintained in DMEM supplemented with 10% dialyzed FBS, 100 μM non essential amino acids, 1% L-glutamine, 1% antibiotic and antimycotic solution, 500 μg/ml Geneticin, and 2 μM methotrexate in a humidified atmosphere of 95% air and 5% CO₂ at 37° C. Confluent cells in 60-mm plates are incubated overnight with serum-free (SF) medium (HAM F-12, 0.5% sterile BSA, and 1% antibiotic and antimycotic). Inhibitors are subsequently added at various concentrations in fresh SF medium for 1 hour. Cells are then stimulated with 10 nM IGF-1 for 1 minute. After treatment, cells are washed twice with ice-cold PBS, harvested, and lysed with fresh lysis buffer (25 mM Tris pH 8.0, 2 mM EDTA pH 8.0, 140 mM NaCl, 1% NP-40, 10.mu.g/ml aprotinin, 20.mu.M phenylmethylsulphonyl fluoride (PMSF), 10 μg/ml Leupeptin and 10 mM orthovanadate) for 1 hour at 4° C., after which the lysates are cleared by centrifugation at 12,000×g for 10 minutes. Protein concentrations of the postnuclear supernatants are determined by the Bradford method (Bio-Rad). To measure tyrosine phosphorylation of the β-subunits of the IGF-1 receptors, lysates are incubated overnight at 4° C. with 2 μg of anti-IGF IR antibody (C-20; Santa Cruz Biotechnology) and 50 μl of 50% protein-A agarose slurry. After 3 washes with lysis buffer, pellets are resuspended in SDS-PAGE sample buffer and boiled for 3 minutes. Proteins are resolved by SDS-PAGE (7.5%) and transferred by electroblotting onto PVDF membranes. Tyrosine phosphorylated receptors are detected by immunoblotting with anti-phosphotyrosine antibody (4G10, Upstate Biotechnology) and then stripped and reprobed with anti-Jak, particularly the Jak JH2, and more particularly the Jak2 V617F or fragment thereof antibody. Detection was with the ECL method (Amersham). As indicated above, parallel experiments may be performed in which IGFR substrate are specifically immunoprecipitated, immunoblotted, and probed with anti-phosphotyrosine antibody.

In another embodiment, agents that modulate (e.g., decrease) the activity of the Jak, particularly the Jak JH2, and especially the Jak2 V617F are identified/confirmed in an animal model. Examples of suitable animals include, but are not limited to, mice, rats, rabbits, monkeys, guinea pigs, dogs and cats. Preferably, the animal used provides an animal model system for a neoplasia disorder associated with altered or aberrant Jak, particularly the Jak JH2, and more particularly the Jak2 V617F or fragment thereof activity. In accordance with this embodiment, the test compound or a control compound is administered (e.g., orally, rectally or parenterally such as intraperitoneally or intravenously) to a suitable animal and the effect on the level of Jak, particularly the Jak JH2, and more particularly the Jak2 V617F or fragment thereof activity is determined.

### Exemplary Methods of Use for the Jak Domain Binding Agents

The invention provides for treatment of disorders ameliorated by administration of a therapeutic compound identified using the method of the invention. Such compounds include but are not limited to proteins, peptides, protein or peptide derivatives or analogs, antibodies, nucleic acids, and small molecules.

The invention provides methods for treating (therapeutically and prophylactically) Jak, particularly the Jak JH2, and more particularly the Jak2 V617F or fragment thereof related neoplasias, comprising administering to a subject an effective amount of a compound identified by a method of the invention. In a preferred aspect, the compound is substantially purified (e.g., substantially free from substances that limit its effect or produce undesired side-effects). The subject is preferably an animal, including but not limited to animals such as cows, pigs, horses, chickens, cats, dogs, etc., and is preferably a mammal, and most preferably human. In a specific embodiment, a non-human mammal is the subject.

As used herein, the term “treating” refers to both therapeutic treatment and prophylactic or preventative measures. Those in need of treatment include those subjects or patients exhibiting symptoms of the disorder, as well as those subjects that are predisposed to the disorder, or diagnosed with the disorder in the absence of symptoms (asymptomatic patients), or in whom the disorder is to be prevented. Consecutive treatment or administration refers to treatment on at least a daily basis without interruption for one or more days. Intermittent treatment or administration, or treatment or administration in an intermittent fashion, refers to treatment that is not consecutive, but rather cyclic in nature. The treatment regime herein can be either consecutive or intermittent. Subjects for whom the preventive measures are appropriate include those with one or more known risk factors for a Jak, particularly the Jak JH2, and more particularly the Jak2 V617F-related disorder, such as cancer.

A “disorder” is any condition caused, mediated, exacerbated by, or associated with Jak, particularly the Jak JH2, and more particularly the Jak2 V617F activity that would benefit from treatment with modulators identified using the methods of the present invention. Such conditions include neoplasias.

The term “effective amount” refers to an amount of a small molecule modulator effective to treat a disease or disorder in a mammal. In the case of cancer, the effective amount of a modulator (i.e., an inhibitor) may reduce the number of cancer cells; reduce tumor size; reduce cancer cell infiltration into peripheral organs; reduce tumor metastasis; and/or relieve one or more of the symptoms associated with the disorder. In cancer therapy, for example, in vivo efficacy can be measured by assessing the time to disease progression (TTP) and/or determining the response rate (RR).

Formulations and methods of administration that can be employed when the compound comprises a nucleic acid are described above; additional appropriate formulations and routes of administration are described below.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu (1987) J. Biol. Chem. 262:4429-4432), construction of a nucleic acid as part of a retroviral or other vector, etc. Methods of introduction can be enteral or parenteral and include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system (CNS) by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally, e.g., by local infusion during surgery, topical application, e.g., by injection, by means of a catheter, or by means of an implant, said implant being of a porous, non-porous, or gelatinous material, including membranes, such as sialastic membranes, or fibers. In one embodiment, administration can be by direct injection into cerebrospinal fluid (CSF) or at the site (or former site) of hyperproliferative cells in CNS tissue.

In another embodiment, the compound can be delivered in a vesicle, in particular a liposome (see Langer, Science 1990; 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In yet another embodiment, the compound can be delivered in a controlled release system. In one embodiment, a pump may be used (see Langer, supra; Sefton CRC Crit. Ref. Biomed. Eng. 1987; 14:201; Buchwald et al. Surgery 1980; 88:507; Saudek et al., N. Engl. J. Med. 1989; 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 1983; 23:61; see also Levy et al. Science 1985; 228:190; During et al. Ann. Neurol. 1989; 25:351; Howard et al. J. Neurosurg. 1989; 71:105). In yet another embodiment, a controlled release system can be placed in proximity of the therapeutic target.

Other controlled release systems are discussed in the review by Langer (1990, Science 249:1527-1533).

### Pharmaceutical Compositions

The present invention also provides pharmaceutical compositions. Such compositions comprise a therapeutically effective amount of an agent, and a pharmaceutically acceptable carrier. In a particular embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the subject. The formulation should suit the mode of administration.

In a preferred embodiment, the composition is formulated in accordance with routine procedures as a pharmaceutical composition adapted for intravenous administration to a human. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachet indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of a compound of the invention which will be effective in the treatment of a hyperproliferative disorder can be determined by standard clinical techniques based on the present description. In addition, in vitro assays may optionally be employed to help identify optimal dosage ranges. The precise dose to be employed in the formulation will also depend on the route of administration, and the seriousness of the disease or disorder, and should be decided according to the judgment of an attending physician and the patient's condition. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Suppositories generally contain active ingredient in the range of 0.5% to 10% by weight; oral formulations preferably contain 10% to 95% active ingredient.

### Nucleic Acids

The invention provides methods for identifying agents capable of binding the Jak, particularly the Jak JH2, and more particularly the Jak2 V617F to modulate (e.g., inhibit) the Jak, particularly the Jak JH2, and more particularly the Jak2 V617F.

In one embodiment, a nucleic acid comprising a sequence encoding a peptide or protein capable of inhibiting the Jak, particularly the Jak JH2, and more particularly the Jak2 V617F is administered. Any suitable methods for administering a nucleic acid sequence available in the art can be used according to the present invention.

In an alternate embodiment, a nucleic acid comprising a sequence encoding a peptide or protein capable of activating the Jak, particularly the Jak JH2, and more particularly the Jak2 V617F or maintaining the Jak, particularly the Jak JH2, and more particularly the Jak2 V617F in an activated state or conformation is administered. Any suitable methods for administering a nucleic acid sequence known in the art can be used according to the present invention.

Methods for administering and expressing a nucleic acid sequence are generally known in the area of gene therapy. For general reviews of the methods of gene therapy, see Goldspiel et al. Clinical Pharmacy 1993; 12:488-505; Wu, et al., Biotherapy 1991; 3:87-95; Tolstoshev gAnn. Rev. Pharmacol. Toxicol. 1993; 32:573-596; Mulligan, Science 1993; 260:926-932; and Morgan, et al., Ann. Rev. Biochem. 1993; 62:191-217; May (1993) TIBTECH 11(5): 155-215. Methods commonly known in the art of recombinant DNA technology that can be used in the present invention are described in Ausubel et al. (eds.), 1993, Current Protocols in Molecular Biology, John Wiley & Sons, NY; and Kriegler (1990) Gene Transfer and Expression, A Laboratory Manual, Stockton Press, NY.

In a particular aspect, a compound comprises a nucleic acid encoding a peptide or protein capable of competitively binding to the Jak, particularly the Jak JH2, and more particularly the Jak2 V617F and inhibiting its activity, such nucleic acid being part of an expression vector that expresses the peptide or protein in a suitable host. In particular, such an expression vector has a promoter operably linked to the coding region, said promoter being inducible or constitutive (and, optionally, tissue-specific). In another particular embodiment, a nucleic acid molecule is used in which the coding sequences and any other desired sequences are flanked by regions that promote homologous recombination at a desired site in the genome, thus providing for intrachromosomal expression of the nucleic acid (Koller, et al. Proc. Natl. Acad. Sci. USA 1989; 86:8932-8935; Zijlstra et al. Nature 1989; 342:435-438).

Delivery of the nucleic acid into a subject may be direct, in which case the subject is directly exposed to the nucleic acid or nucleic acid-carrying vector; this approach is known as in vivo gene therapy. Alternatively, delivery of the nucleic acid into the subject may be indirect, in which case cells are first transformed with the nucleic acid in vitro and then transplanted into the subject, known as “ex vivo gene therapy”.

In another embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286); by direct injection of naked DNA; by use of microparticle bombardment (e.g., a gene gun; Biolistic, Dupont); by coating with lipids, cell-surface receptors or transfecting agents; by encapsulation in liposomes, microparticles or microcapsules; by administering it in linkage to a peptide which is known to enter the nucleus; or by administering it in linkage to a ligand subject to receptor-mediated endocytosis (see, e.g., Wu, et al., J. Biol. Chem. 1987; 262:4429-4432), which can be used to target cell types specifically expressing the receptors. In another embodiment, a nucleic acid-ligand complex can be formed in which the ligand comprises a fusogenic viral peptide that disrupts endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992 (Wu et al.); WO 92/22635 dated Dec. 23, 1992 (Wilson et al.); WO92/20316 dated Nov. 26, 1992 (Findeis et al.); WO93/14188 dated Jul. 22, 1993 (Clarke et al.), WO 93/20221 dated Oct. 14, 1993 (Young)). Alternatively, the nucleic acid can be introduced intracellularly and incorporated within host cell DNA for expression, by homologous recombination (Koller, et al., Proc. Natl. Acad. Sci. USA 1989; 86:8932-8935; Zijlstra et al., Nature 1989; 342:435-438).

In a further embodiment, a retroviral vector can be used. (See Miller et al. Meth. Enzymol. 1993; 217:581-599) These retroviral vectors have been modified to delete retroviral sequences that are not necessary for packaging of the viral genome and integration into host cell DNA. The nucleic acid encoding the protein to be used in gene therapy is cloned into the vector, which facilitates delivery of the gene into a subject. More detail about retroviral vectors can be found in Boesen et al. (1994) Biotherapy 6:291-302, which describes the use of a retroviral vector to deliver the mdr1 gene to hematopoietic stem cells in order to make the stem cells more resistant to chemotherapy. Other references illustrating the use of retroviral vectors in gene therapy are: Clowes et al. J. Clin. Invest. 1994; 93:644-651; Kiem et al., Blood 1994; 83:1467-1473; Salmons, et al., Human Gene Therapy 1993; 4:129-141; and Grossman, et al., Curr. Opin. in Genetics and Devel. 1993; 3:110-114.

Other viral vectors, including adenoviruses, may be used in gene therapy. Adenoviruses are especially attractive vehicles for delivering genes to respiratory epithelia. Adenoviruses naturally infect respiratory epithelia, the infection of which results in a mild respiratory disease. Other targets for adenovirus-based delivery systems are the liver, central nervous system, endothelial cells, and muscle. Moreover, adenoviruses have the advantage of being capable of infecting non-dividing cells. Kozarsky and Wilson (1993) Current Opinion in Genetics and Development 3:499-503 present a review of adenovirus-based gene therapy. Bout et al. Human Gene Therapy 1994; 5:3-10 demonstrated the utility of adenovirus vectors for introducing genes into the respiratory epithelia of rhesus monkeys. Other instances pertaining to the use of adenoviruses in gene therapy can be found in Rosenfeld et al., Science 1991; 252:431-434; Rosenfeld et al. Cell 1992; 68:143-155; Mastrangeli et al. J. Clin. Invest. 1993; 91:225-234; PCT Publication WO94/12649; and Wang, et al. Gene Therapy 1995; 2:775-783. Adeno-associated virus (AAV) has also been proposed for use in gene therapy (Walsh et al. Proc. Soc. Exp. Biol. Med. 1993; 204:289-300; U.S. Pat. No. 5,436,146).

Another suitable approach to gene therapy involves transferring a gene to cells in tissue culture by methods such as, for example, viral infection or electroporation-mediated, liposome-mediated, or calcium phosphate-mediated transfection. Usually, the method of transfer also includes the transfer of a selectable marker into the cells. The cells are then placed under selection to isolate those cells that have been productively transfected. Such selected cells are then delivered to a subject.

In this embodiment, the nucleic acid is introduced into a cell prior to administration of the resulting recombinant cell in vivo. Such introduction can be carried out by any method known in the art, including, but not limited to, transfection, microinjection, infection with a viral or bacteriophage vector comprising the desired nucleic acid sequences, cell fusion, chromosome-mediated gene transfer, microcell-mediated gene transfer, and spheroplast fusion. Numerous techniques are known in the art for the introduction of foreign genes into cells (see, e.g., Loeffler et al., Meth. Enzymol. 1993; 217:599-618; Cohen et al. Meth. Enzymol. 1993; 217:618-644; Cline, Pharmac. Ther. 1985; 29:69-92) and may be used in accordance with the present invention, provided that the necessary developmental and physiological functions of the recipient cells are not disrupted. Such a technique provides for the stable transfer of the nucleic acid to the cell, so that the nucleic acid is expressible by the cell and preferably heritable and expressible by its cell progeny.

The resulting recombinant cells can be delivered to a subject by various methods known in the art. In a preferred embodiment, epithelial cells are injected, e.g., subcutaneously. In another embodiment, recombinant skin cells may be applied as a skin graft onto the subject; recombinant blood cells (e.g., hematopoietic stem or progenitor cells) are preferably administered intravenously. The amount of cells envisioned for use depends on the desired effect, the condition of the subject, etc., and can be determined by one skilled in the art.

Cells into which a nucleic acid can be introduced for purposes of gene therapy encompass any desired, available cell type, and include but are not limited to hepatocyte cells, muscle cells, glial cells (e.g., oligodendrocytes or astrocytes), epithelial cells, endothelial cells, keratinocytes, and fibroblasts; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood or fetal liver. In a preferred embodiment, a cell used for gene therapy is autologous to the treated subject.

In an embodiment in which recombinant cells are used in gene therapy, a nucleic acid encoding an agent (e.g., a peptide or protein) capable of modulating the activity of a Jak is introduced into cells such that it is expressible by the cells and/or their progeny, and the recombinant cells are then administered in vivo for therapeutic effect. In a specific embodiment, stem or progenitor cells are used. Any stem or progenitor cells which can be isolated and maintained in vitro can be used in accordance with this embodiment of the present invention (see e.g. PCT Publication WO 94/08598, dated Apr. 28, 1994; Stemple, et al., Cell 1992; 71:973-985; Rheinwald, Meth. Cell Bio. 1980; 21A:229; and Pittelkow, et al., Mayo Clinic Proc. 1986; 61:771).

In another embodiment, the nucleic acid to be introduced for purposes of gene therapy may comprise an inducible promoter operably linked to the coding region, such that expression of the nucleic acid is controllable by regulating the presence or absence of the appropriate inducer of transcription.

Direct injection of a DNA coding for a peptide or protein capable of binding to the IGF1RK domain of the IGF1 receptor and modulating its activity may also be performed according to, for example, the techniques described in U.S. Pat. No. 5,589,466. These techniques involve the injection of “naked DNA”, i.e., isolated DNA molecules in the absence of liposomes, cells, or any other material besides a suitable carrier. The injection of DNA encoding a protein and operably linked to a suitable promoter results in the production of the protein in cells near the site of injection and potentially the elicitation of an immune response in the subject to the protein encoded by the injected DNA.

### Kits

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

### DESCRIPTION OF THE PREFERRED EMBODIMENTS

The following examples are set forth so as to provide those of ordinary skill in the art with a complete disclosure and description of how to make and use the assay, screening, and therapeutic methods of the invention, and are not intended to limit the scope of the invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

Crystal structures of both JH2-WT and -VF with bound Mg-ATP, in the same crystal lattice are provided to compare the differences in structure induced by V617F (presented in Preliminary Studies). These constructs start at V536 and therefore do not include the JH2 autophosphorylation site S523 in the SH2-JH2 linker. Baculoviruses that encode residues 513-812 (JH2-WT[513]), and protein expression levels in Sf9 cells are similar (i.e., excellent) to those of the 536-812 constructs are available. Crystals and structures of JH2-WT(513) and -VF(513) are provided. Assuming that autophosphorylation of S523 by JH2 occurs in cis, we can visualize S523 bound in the JH2-WT active site. Such a structure would reveal which residues proximal to S523 are important in substrate recognition and whether the SH2-JH2 linker, to which residues 513-536 belong, interacts or not with αC. This latter point is important in understanding why JH2 autophosphorylation of S523 is critical for maintaining a low basal activity of Jak2.

JH2-WT(513) and -VF(513), containing the three surface-hydrophobic mutations were purified in their unphosphorylated and S523-phosphorylated forms (FIG. 4). Both forms of JH2-WT(513) will be crystallized to visualize S523 (unphosphorylated) in the active site of JH2. Once S523 is phosphorylated, it should be expelled from the active site, and this phosphorylated segment may or may not adopt an ordered conformation. If it does adopt an ordered conformation, a crystal structure of the phosphorylated form could reveal the basis for the requirement of S523 phosphorylation for maintenance of the basal state of Jak2.

Similarly, unphosphorylated JH2-VF(513) will be crystallized to determine whether the structure can shed light on the apparent inability of JH2-VF to phosphorylate S523. Not known is whether S523 binds in the active site of JH2-VF but is not phosphorylated, or whether S523 does not bind in the active site, possibly due to the structural alterations in αC induced by the mutation (see FIG. 3b).

JH2-WT(513) will be used to determine whether S523 is autophosphorylated in cis or in trans. We will incubate purified JH2-WT(513) with Mn-ATP (Mn²⁺ is more efficacious than Mg²⁺) at various protein concentrations and for various times and monitor phosphorylation of the tryptic peptide containing S523/pS523 by MALDI-TOF mass spectrometry. If the phosphorylation content of S523 is linear with JH2-WT(513) concentration, then the data is consistent with cis-autophosphorylation. If the dependence of S523 goes as the square of the protein concentration, trans-autophosphorylation of S523 would be indicated. An intermediate result would indicate that cis-autophosphorylation can occur to some extent, and we will examine more closely the concentration-dependence at low protein concentration, where trans-autophosphorylation would be less favored. We will also measure the K_{d} of ATP binding to JH2-WT(513) and -VF(513), to determine whether the SH2-JH2 linker (containing S523) affects the ATP binding properties.

### Methods

For both crystallization projects (JH2 and JH2-JH1), a Mosquito crystallization robot will be used for crystal-lization trials, using the various commercial crystallization screening kits available, as well as our own. The in-house x-ray diffraction system consists of a Rigaku MicroMax-007 rotating anode with Osmic Confocal Blue optics and an R-AXIS IV++ image plate detector. For high-resolution data collection, we have extensive experience at synchrotron beam lines X4A/C, X12C, and X25 at the National Synchrotron Light Source, Brookhaven National Laboratory.

### Example 2

JH21-WT will be crystallized in its unphosphorylated state. Mass spectrometry will be used to characterize the phosphorylation state of JH21-WT in each of the four Source Q elution peaks (FIG. 7b). If the first peak is unphosphorylated JH21-WT, potentially representing the autoinhibited state, this protein will be used directly in crystallization trials. Using limited proteolysis, whether the presence of an ATP analog or commercially available inhibitors of Jak2 JH1 (e.g., CMP6 (Thompson, et al., Bioorg. Med. Chem. Lett. 2002; 12: 1219-1223) from Calbiochem) stabilizes or destabilizes the JH2-JH1 autoinhibitory interaction will be tested. These data will direct which additives to include in crystallization trials of unphosphorylated JH21-WT. Depending on the phosphorylation mapping results, we will consider introducing mutations (Tyr→Phe or Ser→Ala) at these positions (or incubating with protein phosphatases) to obtain larger quantities of unphosphorylated JH2-JH1 for crystallization trials.

JH21-WT and JH21-VF will undergo kinase assays on the two unphosphorylated proteins to determine whether V617F undergoes autophosphorylation on the JH1 activation loop (Y1007/Y1008) more readily than WT, as predicted from the model (FIG. 6). In addition, limited proteolysis will be performed on the two proteins to determine whether wild-type JH21 (“hinged”, state I favored, FIG. 6) is more resistant to proteases than V617F (“unhinged”, state II favored).

Once diffracting crystals of JH21 are obtained, the structure will be solved by molecular replacement, using the crystal structure of JH2 already determined and crystal structures of JH1 available from the Protein Data Bank. If not, we will employ heavy-atom methods, in particular, single-/multi-wavelength anomalous diffraction (SAD/MAD) phasing of selenomethionyl-substituted protein. We (Stiegler, et al., J. Mol. Biol. 2009; 393: 1-9) and others (Cronin, et al., Protein Sci. 2007; 16: 2023-2029) have expressed selenomethionyl-containing proteins in Sf9 cells for structure determination.

There is compelling evidence that a properly folded JH2 is required for full activity of JH1 which suggests a possible stabilizing interaction between JH2 and phosphorylated JH1. The goal in this Sub-aim is to crystallize either JH21-WT or JH21-VF in its phosphorylated state, in which Y1007 and Y1008 in the JH1 activation loop are phosphorylated. Unlike in the basal (unphosphorylated) state of JH21, in which the conformational equilibrium for WT and VF could be very different [see FIG. 6, state I (WT) vs. state II (VF)], the conformational state of activated JH21 could be similar for WT and VF.

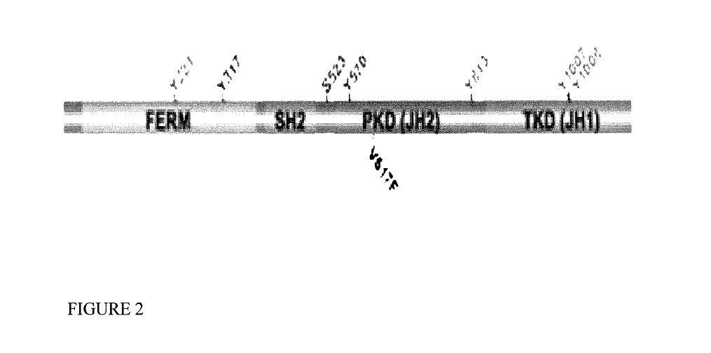

Based on previous phosphorylation mapping studies of Jak2 in cells (see FIG. 2), it is likely that peak 4 in the Source Q elution profile (FIG. 7b) represents JH21 (triply) phosphorylated on Y1007, Y1008, and Y813 (which we will verify by mass spectrometry—Y570 is another possibility). If so, to increase the yield of JH21 phosphorylated on Y1007 and Y1008 only, a Y813A (or Y570A) mutation will be introduced into the JH21 construct and purify JH21 homogeneously phosphorylated on Y1007 and Y1008.

As for unphosphorylated JH21, limited proteolysis will be performed on phosphorylated JH21 in the presence of either Mg-AMPPNP or JH1 inhibitors to see how these small molecules affect the stability of the putative JH2-JH1 complex. These experiments will direct what co-crystallization trials to pursue.

In addition to mapping the sites of autophosphorylation in JH21, the steady-state kinetics (Kₘ, k_{cat}) of JH21-WT versus -VF, both prior to JH1 activation (unphosphorylated) and after activation (phosphorylated on Y1007/Y1008) will be characterized and compared. For these assays, a peptide substrate representing the Y813 autophosphorylation site in Jak2 (there are many known substrates to choose from) will be used. Similar studies for the insulin receptor kinase have been performed (Li, et al., J. Biol. Chem. 2003; 278: 26007-26014), insulin-like growth factor-1 receptor kinase (Favelyukis, et al., Nat. Struct. Biol. 2001; 8: 1058-1063), and muscle-specific kinase (Till, et al., Structure 2002; 10: 1187-1196). In particular, whether, prior to activation, JH21-WT is catalytically less efficient than JH21-VF, as the model (FIG. 6) would predict will be determined, and whether any difference in catalytic efficiency persists after activation of JH1.

The steady-state kinetics of JH21 versus JH1 alone will be studied (which we can express in Sf9 cells). JH1 should have higher catalytic efficiency than JH21 in the basal state (JH1 is autoinhibited by JH2), but lower catalytic activity in the activated state (JH1 is stabilized by JH2 upon activation).

Functional mutagenesis studies of Jak2 in mammalian cells (examples of which are presented in Preliminary Studies) will be performed, monitoring Jak2 phosphorylation on the JH1 activation loop (pY1007/1008), which is a measure of the activity state of Jak2, and on S523, the negative regulatory site phosphorylated by JH2. The cytokine-stimulated phosphorylation of Stat proteins, which are phosphorylated by activated Jak2, will be monitored.

The hypotheses for JH1 regulation by JH2 in Jak2 are:

(i) An autoinhibitory interaction exists between JH2 and JH1 in the basal, unphosphorylated state.
(ii) Phosphorylation of S523 and Y570 by JH2 negatively regulates Jak2 (JH1) through an unknown mechanism(s).
(iii) The structural integrity of 1112 is critical for full activity of Jak2 and for hyperactivity of MPN-causing mutations such as V617F.

In designing mutagenesis experiments to probe the JH2-mediated regulatory mechanisms in Jak2, we will focus on the relationship between JH2 catalytic activity and Jak2 hyperactivity (caused by MPN mutations) and the relationship between JH2 structural integrity and Jak2 hyperactivity. Further whether it is possible to distinguish between MPN-causing mutations in JH2 or JH1 that substitute residues in the JH2-JH1 interface (direct) versus those that cause conformational perturbations that disrupt the interaction (indirect) will be investigated.

Based on the crystal structures of JH2-WT and -VF, two mutations will be introduced into JH2, F594A and L583V (FIG. 3b). F594 is adjacent to F595 in αC and its position is affected by V617F. F595A suppresses the hyperactivity of V617F (FIG. 10a), and we will test whether F594A will also suppress V617F and restore S523 phosphorylation. If so, it suggests a causal structural linkage from F617 (mutation site) to K581 (catalytic residue) through F595 and F594.

In the structures of JH2-WT and -VF, L583 is observed to change side-chain conformers (FIG. 3b). As another test of the importance of F594 in the constitutive activity of V617F, the mutation L583V into Jak2 will be introduced. V583 (branched at Cβ) should force F594A into the position observed in JH2-VF. If L583V is hyperactive, comparable to V617F, it would provide further evidence (in addition to V617F/F594A) that the altered position of F594 is a critical aspect of the hyperactivity of V617F.

To determine whether Jak2 containing a structurally compromised JH2 can be fully activated or not, the mutation F739R into JH2 will be introduced. F739 is in the middle of the hydrophobic core of the C lobe. If F739R cannot be activated, it would indicate that a structurally intact JH2 is required for Jak2 activity and suggest, along with the V617F suppression data, that MPN mutations partially destabilize JH2.

While most of the activating mutations in Jak2 have been mapped to the pseudokinase domain (JH2), several have been mapped to the tyrosine kinase domain (JH1) (Haan, et al. J. Cell. Mol. Med. 2010; 14: 504-527). Two of these mutations (D873N, T875N) are found in the β2-β3 loop of JH1. Residues in this loop, which is distal to the active site, would not be predicted to affect catalytic activity, and therefore these JH1 residues are prime candidates to be in the JH2-JH1 interface. These two mutations will be individually introduced into Jak2 and their phosphorylation levels assessed relative to wild-type Jak2 and V617F. Assuming the mutants are found to be hyperactivated, the tyrosine kinase domains (JH1) alone will be expressed to see whether these mutations increase the intrinsic catalytic activity of JH1, which would be an alternative explanation for their hyperactivity.

### Example 3

### Methods

Protein Expression and Purification.

JH2 from JAK2 was amplified and cloned into pFASTBAC1 vector (Invitrogen) with a Thrombin-cleavable N-terminus GST-tag or a C-terminus 6×His-tag and expressed as a fusion protein in insect cells (Sf9) cells. For protein expression, cells were infected with 10% (v/v) virus supernatant and grown for 48 h and harvested by centrifugation. Cell pellets containing GST-JH2 or JH2-His fusion protein were resuspended in lysis buffer containing 20 mM TRIS-HCl, pH 8.5, 500 mM NaCl, 15% (v/v) glycerol, 0.5 mM TCEP and 20 mM imidazole (for JH2-His protein only), supplemented with protease inhibitors cocktail (Roche), lysed using cell-disruptor (Avensis) and clarified by centrifugation for 1 h at 45000×g. The supernatant was incubated for 2 h with pre-washed GST beads (GE Healthcare) or Ni-NTA beads (Qiagen) with gentle rotation at 4° C. The beads were extensively washed and the fusion protein was eluted with 10 mM glutathione (Sigma-Aldrich) for GST-JH2 or 250 mM imidazole (Fluka) for JH2-His protein. Fractions containing the fusion protein were pooled and dialyzed for 2 h at 4° C. in buffer (20 mM TRIS-HCl, pH 8.5, 250 mM NaCl, 15% (v/v) glycerol and 0.5 mM TCEP). For JH2-His fusion protein, after dialysis samples were incubated with 10 U ml⁻¹ Thrombin (Enzyme Research Laboratory) overnight. Proteins were loaded onto a MonoQ column (GE Healthcare) equilibrated with 20 mM TRIS-HCl, pH 8.5, mM NaCl, 15% (v/v) glycerol and 0.5 mM TCEP and eluted with a linear gradient 1-200 mM NaCl. Fractions containing purified GST-JH2 or JH2 were analyzed by Coomassie staining and pooled and concentrated to 1 mg ml⁻¹ for further use. JAK2 JH1 kinase domain (aa 836-1132) was cloned by PCR amplification into pFASTBAC1 plasmid with an N-terminus GST-tag and purified as previously described (Lucet et al., Blood 107:176-183 (2006)).

Autophosphorylation Reaction.

The autophosphorylation reactions were carried out using 1 μg⁻¹ of JH2, 10 mM ATP (Sigma-Aldrich) or 10 μCi [γ-³²P] ATP (PerkinElmer), 20 mM MnCl₂, 300 mM NaCl, 10% (v/v) glycerol, 0.5 mM TCEP, and 20 mM Tris-HCl (pH 8.0) at room temperature. The reactions were stopped by adding EDTA to a final concentration of 100 mM. The phosphorylation states of JH2 were monitored by autoradiography and native-PAGE (PhastGel System, GE Healthcare) and Western blotting.

Mass Spectrometry.

JAK2 gel bands were processed for in-gel digestion as previously reported (Shevchenko et al., Nat. Protoc. 1:2856-2860 (2006)). Phosphopeptide enrichment was performed with titanium dioxide microcolumns, with eluates desalted with Poros R3 microcolumns as previously described (Thingholm et al., Nat. Protoc. 1:1929-1935 (2006)). LC-MS MS was conducted on an EASY-nLC system (Thermo Fisher Scientific) coupled to an LTQ-Orbitrap XL mass spectrometer (Thermo Fisher Scientific) as previously reported (Ye et al., J. Proteome Res. 9:3561-3573 (2010)) except that chromatography was conducted with a 30 min gradient. Raw data files were submitted for Mascot searches (Matrix Science) using Proteome Discoverer 1.1 software (Thermo Fisher Scientific). Databases containing the human JAK2 protein sequence were searched with the following parameters: ESI-TRAP was selected as the instrument setting, with specified mass tolerances of 10 ppm (precursor) and 0.6 Da (fragment). Serine, threonine and tyrosine phosphorylation, along with methionine oxidation, were set as variable modifications. Cysteine carbamidomethylation was included as a fixed modification and Trypsin-P specified with a maximum of two missed cleavages. Only MS-MS spectra from JAK2 phosphopeptides possessing Mascot ion scores above 20 were manually validated for the sites of phosphorylation.

Mant-ATP Binding Assay.

The fluorescence intensity of mant-ATP (Invitrogen) complex with JH2 was measured using FluoroMax-2 spectrofluorimeter. Mant-ATP (1 μM) was added to a buffer solution (20 mM TRIS-HCl, pH 8, 200 mM NaCl, 10% (v/v) glycerol and 0.5 mM TCEP) along with 5 mM MnCl₂ and 1 μM JH2 (from peak 2 fraction). The excitation and emission wavelengths were 280 nm and 440 nm, respectively, and emission was scanned from 400-500 nm. For kd measurements, increased concentration of purified JH2 (0.25 μM-6 μM) was added to buffer solution with 5 mM MnCl₂ and 1 μM mant-ATP.

Transfection, Western Blotting and Luciferase Assay.

Human JAK2 wild-type, JAK2 JH2 domain and human EpoR were obtained by PCR amplification and cloned with a C-terminus HA-tag into pCI-neo mammalian expression plasmid (Promega). JAK2 mutations were done using QuickChange Site-Directed Mutagenesis method (Stratagene) and verified by sequencing. STAT1 and STATS plasmids were previously described. JAK2-deficient γ 2A cells (fibrosarcoma cells) were transfected with different JAK2 mutants using Fugene (Roche) according to manufacturer's instructions. After 8 h, cells were lysed in lysis buffer (50 mM TRIS-HCl, pH 8, 150 mM NaCl, 100 mM NaF, 10% (v/v) glycerol, 1% (v/v) Triton-X and protease inhibitors cocktail) and protein phosphorylation was analyzed by Immunoprecipitation and Western blotting with anti-phosphotyrosine (4G10) antibody (Millipore), anti-pJAK2 1007-1008 (Cell Signaling Technology), anti-pSer523⁵, anti-pTyr570⁹ and anti-HA (Covance) antibody. STAT1 and STATS phosphorylation was analyzed in γ 2A cells transfected with different JAK2 constructs together with STAT1 or STATS as indicated, and after 8 h cells were starved for 12 h in serum-free media followed by stimulation with hIFN-γ (100 U ml⁻¹, R&D Systems) or hEpo (50 U ml⁻¹, Janssen-Cilag). After cell lysis, STAT1 phosphorylation was analysed by Western Blotting with anti-pSTAT1 antibody or anti-pSTATS antibody (Cell Signalling Technology). STAT1 and STATS transcriptional activity of JAK2 wild type and K581A mutant was measured in γ 2A cells using GAS-luc STAT1 reporter or or SPI-luc2 STATS reporter as previously described. After stimulations, cells were lysed in 1× reporter lysis buffer (Promega). Luminescence was recorded using Luminoscan Ascent 96-well plate luminometer (Thermo Labsystem) and the transfection efficiency was normalized using γ-GAL values.

### Results

### Phosphorylation of Purified JAK2 JH2 In Vitro

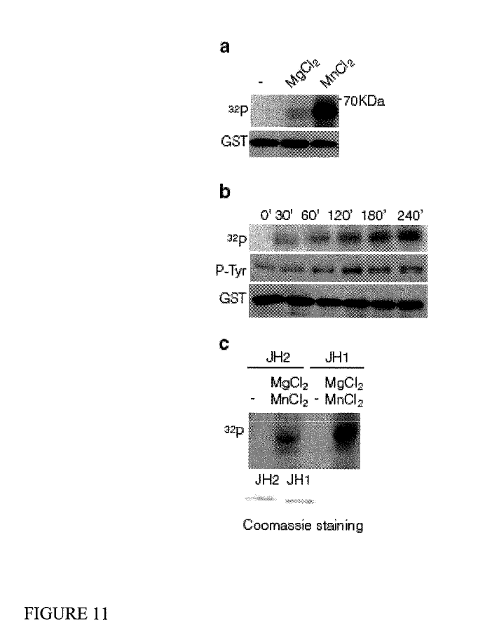

JH2 was expressed as a GST-fusion protein in insect cells (Sf9). Purified GST-JH2 was used in an in vitro kinase assay, which showed a time-dependent phosphorylation of JH2 with a strong preference for Mn2⁺ as divalent cation (FIGS. 11a and b). A comparison of the autophosphorylation activity of purified JH1 versus JH2 of JAK2 indicates that JH2 has ˜10% of JH1 activity (FIG. 11c), which could explain why JH2 activity has previously gone unnoticed. To verify the autophosphorylation activity of JH2, a kinase-inactivating mutation, K581A, was introduced in JH2. This lysine (in γ-strand 3 of the JH2 N-lobe) serves to coordinate the α- and γ-phosphates of ATP in active protein kinases (Hubbard, et al., Annu. Rev. Biochem. 2000; 69: 373-398). GST-JH2 wild-type (WT) and K581A mutant were produced and purified side-by-side from insect cells. In vitro kinase assay shows that the kinase-inactive JH2 mutant is devoid of autophosphorylation activity.

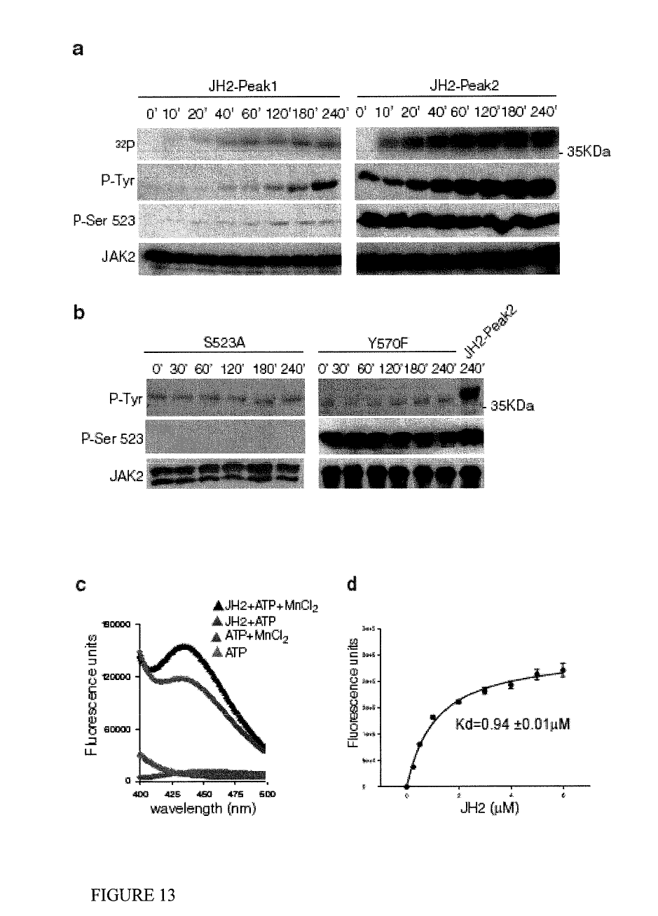

To further confirm that the observed kinase activity was due to JH2 autophosphorylation and not to phosphorylation by a contaminating protein kinase, JH2 was in vitro translated and analyzed in a kinase assay. Western blotting showed that in vitro translated JH2 of JAK2 is autophosphorylated on tyrosine. Next, JH2 wild-type (WT) and JH2 K581A were in vitro translated, His-tag purified and subjected to an in vitro kinase assay in the presence of [γ-³²P] ATP. Autophosphorylation was detected in JH2 domain but not in JH2 K581A mutant (Supplementary FIG. 13c). Taken together, these results demonstrate that JH2 possesses autophosphorylation activity.

### Purified JAK2 JH2 Becomes Autophosphorylated on Ser523 and Tyr570 Residues

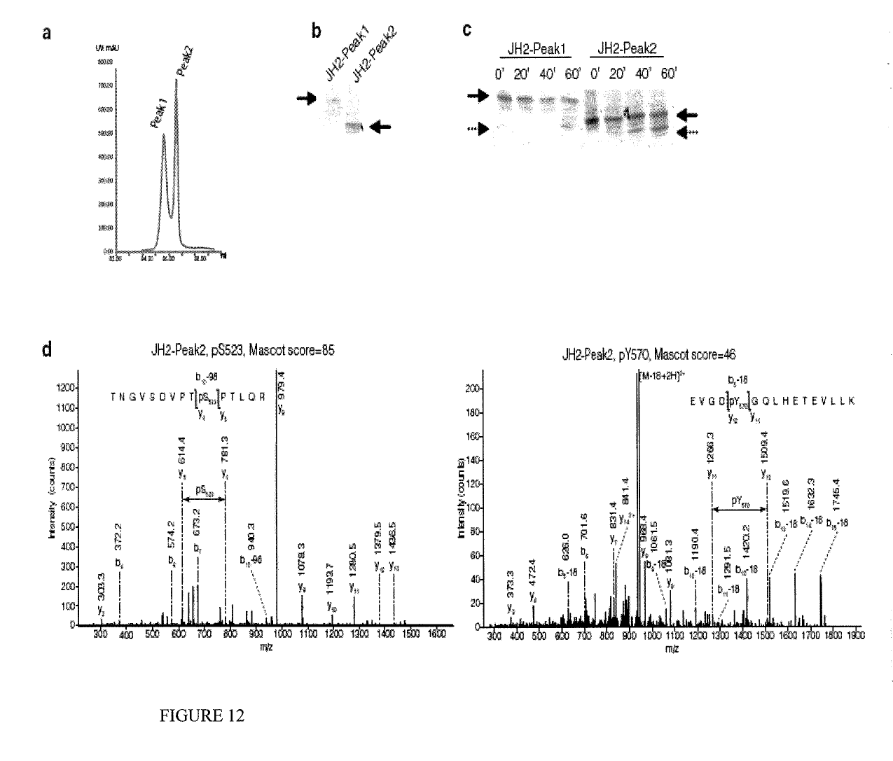

To study the kinase activity of the JAK2 pseudokinase domain in more detail, His-tagged JH2 was expressed in insect cells and purified using Ni-NTA affinity and anion-exchange chromatography. JH2 eluted in two closely spaced peaks on an anion-exchange column (FIG. 12a). In native-gel electrophoresis, JH2 in peak 2 (JH2-Peak2) migrated faster than JH2-Peak1 (FIG. 12b). The chromatography and electrophoresis data are suggestive of a higher phosphorylation state for JH2-Peak2 than for -Peak1. The autophosphorylation activities of the two JH2 samples were analyzed in an in vitro kinase assay. Native-gel electrophoresis showed the appearance of a faster-migrating band for both samples at later time points of the reaction, consistent with an increase in phosphorylation state (FIG. 12c). LC-ESI-MS and MS LTQ-Orbitrap mass spectrometry was used to identify the phosphorylated residues in JH2. The analysis showed that JH2-Peak1 was unphosphorylated at time zero and underwent autophosphorylation on Ser523 during the kinase reaction (data not shown). In contrast, JH2-Peak2 was robustly (stoichiometrically) phosphorylated on Ser523 at time zero, hence explaining the migration difference between the proteins in the two peaks, and became phosphorylated additionally on Tyr570 during the kinase reaction (FIG. 12d).

Further analysis of the JH2 autophosphorylation activity in kinase assays demonstrated that JH2-Peak2 has substantially higher tyrosine kinase activity than JH2-Peak1 (FIG. 13a). To investigate the basis for this difference, the phosphorylation state of Ser523 was monitored by Western blotting using an anti-pSer523 specific antibody (Ishida-Takahashi, et al., Mol. Cell. Biol. 2006; 26: 4063-4073). Consistent with the mass spectrometry results, Ser523 phosphorylation increased in JH2-Peak1 during the kinase reaction, whereas JH2-Peak2 was fully phosphorylated already at time zero and the pSer523 level remained constant during the reaction (FIG. 13a). Moreover, GST-JH2 K581A mutant purified from insect cells does not show any phosphorylation on Ser523 demonstrating that Ser523 is de facto autophosphorylation site of JH2. These results, together with the results in FIG. 12c, suggest that phosphorylation of Ser523 regulates the tyrosine kinase activity of JH2. To address this possibility, Ser523 was mutated to alanine, and, consistent with the hypothesis, S523A did not undergo tyrosine phosphorylation during an in vitro kinase assay (FIG. 13b). Mutation of Tyr570 to phenylalanine did not affect Ser523 phosphorylation, but abolished tyrosine phosphorylation. These phospho-specific antibody data also confirm the mass spectrometry identification of the two autophosphorylated residues in JH2, Ser523 and Tyr570. Importantly, these two residues have previously been identified as negative regulatory sites in JAK2 (Ishida-Takahashi, et al., Mol. Cell. Biol. 2006; 26: 4063-4073; Mazurkiewicz-Munoz, et al., Mol. Cell. Biol. 2006; 26: 4052-4062; Feener, et al., Mol. Cell. Biol. 2004; 24: 4968-4978; Argetsinger, et al., Mol. Cell. Biol. 2004; 24: 4955-4967).

If the pseudokinase domain of JAK2 is an active protein kinase, utilizing ATP as a phosphate donor, it should bind ATP with a physiologic K_{d} value. The binding of ATP to JH2 of JAK2 was evaluated using the fluorescent ATP-analogue mant [2′-(3′)—O—(N-methylanthraniloy)]-ATP. The fluorescence emission scan showed a peak at ˜440 nm only when MnCl₂ and JH2 were present along with mant-ATP (FIG. 13c). Mant-ATP bound to JH2 with a K_{d} of ˜1 μM (FIG. 13d), which is ˜10% of the reported K_{d} for JH1 of JAK2 (Hall, et al., Protein Expr. Purif. 2010; 69: 54-63). Taken together, the in vitro data demonstrate that the pseudokinase domain of JAK2 is a dual-specificity serine and tyrosine kinase. Autophosphorylation of Ser523 is the primary event in JH2 activation, which enhances subsequent autophosphorylation of Tyr570.

### Analysis of JAK2 JH2 in Mammalian Cells

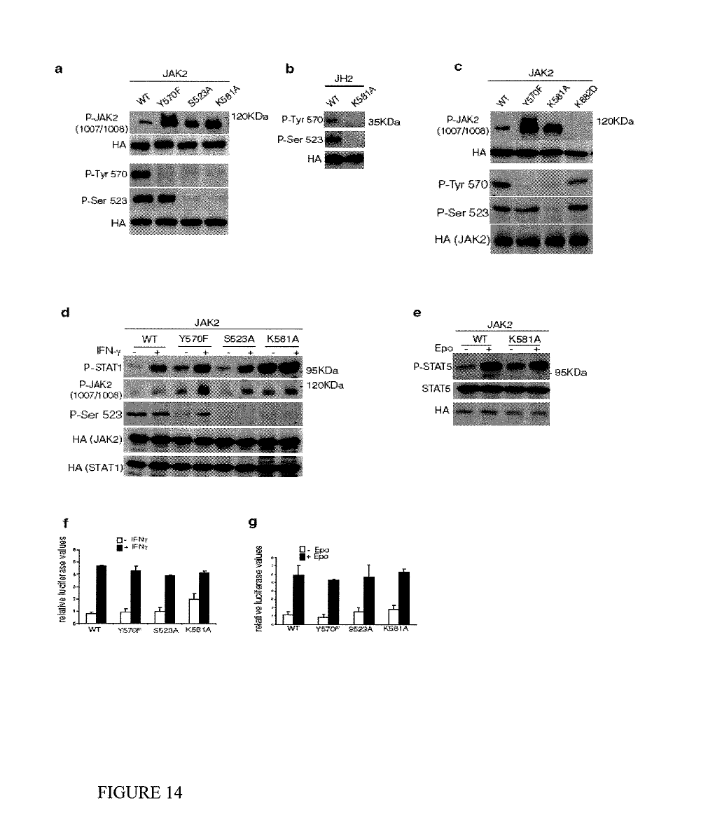

To analyze the function of the catalytic activity of JH2 in a cellular context, the kinase-inactivating point mutation in JH2, K581A, was introduced into JAK2. Various JAK2 constructs were expressed in JAK2-deficient γ 2A cells, and JAK2 phosphorylation was analyzed by Western blotting. JAK2 WT was tyrosine phosphorylated at a low level, and consistent with previous studies (Ishida-Takahashi, et al., Mol. Cell. Biol. 2006; 26: 4063-4073; Mazurkiewicz-Munoz, et al., Mol. Cell. Biol. 2006; 26: 4052-4062; Feener, et al., Mol. Cell. Biol. 2004; 24: 4968-4978; Argetsinger, et al., Mol. Cell. Biol. 2004; 24: 4955-4967), mutation of either Ser523 or Tyr570 increased JAK2 tyrosine phosphorylation of the JH1 activation loop (Tyr1007-Tyr1008), an indicator of JAK2 activation. Similar to the data for S523A and Y570F, JAK2 K581A displayed a higher level of tyrosine phosphorylation than JAK2 WT and, importantly, Ser523 and Tyr570 sites were not phosphorylated in K581A (FIG. 14a). In addition, these results corroborate the in vitro results (FIG. 13b) and show that, in cells, phosphorylation of Tyr570 is dependent on Ser523 phosphorylation of JH2.

To confirm the role of JH2 catalytic activity in phosphorylation of Ser523 and Tyr570, JH2 alone, WT or the K581A mutant, were expressed in □2A cells. In JH2 WT, both Ser523 and Tyr570 were phosphorylated, and the K581A mutation abolished their phosphorylation (FIG. 14b). Moreover, in the context of full-length JAK2 bearing a point mutation that abrogates JH1 activity (K882D), phosphorylation of Ser523 occurred to the same extent as in JAK2 WT, and K882D mutation did not markedly affect phosphorylation of Tyr570 (FIG. 14c). Finally, in JAK2 constructs lacking the entire JH1 domain (JAK2del.JH1) phosphorylation of Ser523 and Tyr570 occurred to the same level as in JAK2 WT. Finally, we wanted to verify that the effects of the K581A mutation were due to abrogation of JH2 catalytic activity and minimize the possibility that they were caused by secondary conformational alterations in JH2. To this end, a more conservative mutation, K581R, and, separately, a distinct inactivating mutation, N678A (catalytic loop) were introduced into the full length protein. The K581R mutant showed clear decreases in Ser523 and Tyr570 phosphorylation and increase in JAK2 Y1007-1008 phosphorylation. Similar effects, albeit less pronounced, were observed with N678A mutant. These in-cell data substantiate the conclusion that JH2 is a dual-specificity protein kinase that autophosphorylates Ser523 and Tyr570.

### JH2 Catalytic Activity is Required to Maintain Low Basal Activity of JAK2

JH2 activity was next investigated in cytokine receptor-mediated signaling by analyzing STAT activation in γ 2A cells in response to cytokine stimulation. Compared to JAK2 WT, the JAK2 mutants S523A, Y570F and K581A showed increased basal phosphorylation of STAT1, but the mutations did not influence the IFN-γ-induced STAT1 phosphorylation (FIG. 14d). There was some variation in the level of STAT1 phosphorylation between the experiments (FIG. 14d), but increased basal phosphorylation of STAT1 was a consistent finding that was also observed with K581A in EpoR-induced STATS phosphorylation (FIG. 14e). The JH2 activity was investigated in cytokine-induced transcriptional response by using reporter-gene analysis. Introduction of K581A in JAK2 increased the basal STAT1- and STATS-dependent reporter-gene activation, but did not affect the IFN-γ or Epo induced responses (FIGS. 4f and g). Taken together, these results indicate that JH2 catalytic activity is required to maintain a low basal level of JAK2 (JH1) activity.

### JAK2 MPN-Causing Mutations Affect the Catalytic Activity of JH2

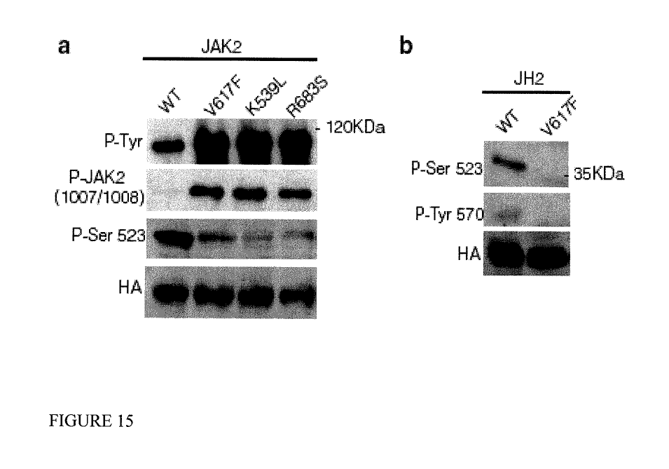

Our results showing that the catalytic activity of JH2 regulates the basal activity of JAK2 raises the question of the possible connection of this activity to human JAK2 mutants and disease pathogenesis. We were interested in understanding whether the catalytic activity of JH2 was involved in the pathogenesis of JAK2 MPN mutants. For this analysis, we chose three distinct MPN-causing JH2 mutants: V617F (exon 14, predominant MPN-causing mutation), K539L (exon 12) (Scott, et al., N. Engl. J. Med. 2007; 356: 459-468) and R683S (exon 16) (Bercovich, et al., Lancet 2008; 372: 1484-1492). Consistent with previous studies, these mutants showed high levels of tyrosine phosphorylation and activation of JAK2 in γ 2A cells when compared to JAK2 WT (FIG. 15a). Interestingly, all three mutants showed significantly decreased Ser523 phosphorylation. These results suggest that the JH2 mutations that cause MPN reduce or abrogate JH2 catalytic activity. To test this hypothesis directly, JH2 alone and its V617F counterpart were analyzed in γ 2A cells. The results show that V617F, like K581A, abrogates Ser523 and Tyr570 phosphorylation (FIG. 15b). Finally, it was determined whether the altered JH2 function is also observed in clinical samples from MPN patients, and thus could be a causative mechanism for the disease. To this end, platelets from three MPN patients carrying the V617F mutation and from a healthy control were isolated and subjected to Tpo stimulation (Supplementary Methods). As a readout for JH2 activity, the phosphorylation of JAK2 Tyr570 was analyzed. Tpo stimulation readily induced Tyr570 phosphorylation in control cells, while in patient samples Tyr570 phosphorylation was significantly reduced, and the reduction correlated with the V617F allelic burden of the patient cells. Taken together, these results show that MPN-causing mutations disturb the catalytic activity of JH2 and abrogate phosphorylation of negative regulatory residues that lead to increased basal activation of JAK2.

### Discussion

Protein kinases have been classified as pseudokinases if they lack conserved residues thought to be required for phosphoryl transfer, and if catalytic activity has not been detected (Boudeau, et al., Trends Cell Biol. 2006; 16: 443-452; Zeqiraj, et al., Curr. Opin. Struct. Biol. 2010; 20: 772-781). Recent studies have provided important new information and insights into the functions of this protein family. Some of the structurally characterized proteins such as VRK3, is unable to bind ATP and obtains a pseudoactive conformation by filling the ATP binding pocket by hydrophobic residues, and thus is retaining the pseudokinase status (Scheeff, et al., Structure 2009; 17: 128-138). However, for several other proteins the functional status has been overturned, and proteins including CASK, haspin, WNK1, HER3(ErbB3), and STRAD have been shown to have ATP-binding and (or) catalytic activity that can be achieved through non-canonical mechanisms (Mukherjee, et al., Cell 2008; 133: 328-339; Eswaran, et al., Proc. Natl. Acad. Sci. U.S.A. 2009; 106: 20198-20203; Min, et al., Structure 2004; 12: 1303-1311; Shi, et al., Proc. Natl. Acad. Sci. U.S.A. 2010; 107: 7692-7697; Zeqiraj, et al., Science 2009; 326: 1707-1711). Each of these pseudokinases utilizes a distinct mechanism to carry out its cellular functions. For example, WNK1 compensates for the missing ATP-binding lysine in □-strand 3 by employing instead a lysine residue in the nucleotide binding loop (Min, et al., Structure 2004; 12: 1303-1311). The calcium calmodulin-activated serine-threonine kinase CASK displays atypical catalytic activity in that Mg²⁺ inhibits its activity (Mukherjee, et al., Cell 2008; 133: 328-339). HER3 lacks the catalytic base aspartate and the crystal structure reveal that it assumes an atypical conformation for active kinases, particularly in αC helix and activation segment (Shi, et al., Proc. Natl. Acad. Sci. U.S.A. 2010; 107: 7692-7697; Jura, et al., Proc. Natl. Acad. Sci. U.S.A. 2009; 106: 21608-21613). However, HER3 was found to retain low levels kinase activity and phosphorylate its intracellular region in vitro, but the physiological role of this activity remains to be determined (Shi, et al., Proc. Natl. Acad. Sci. U.S.A. 2010; 107: 7692-7697). These results argue that each alleged pseudokinase needs to be functionally analyzed and scrutinized for possible catalytic activity. In this study, we have shown that, both in vitro and in cells, the pseudokinase domain of JAK2 is an active dual-specificity protein kinase that phosphorylates two previously identified negative regulatory sites in JAK2, Ser523 and Tyr570. Phosphorylation of these sites is required to maintain low basal activity of JAK2.

These results on the catalytic activity of JH2 provide novel insights into the regulation of JAK activation in signaling by a variety of cytokines such as Epo, Tpo, IFN-γ growth hormone, prolactin, IL-3, IL-5, and GM-CSF. In unstimulated cells, Ser523 has been shown to be the only constitutively phosphorylated residue in JAK2 (Ishida-Takahashi, et al., Mol. Cell. Biol. 2006; 26: 4063-4073), and phosphorylation of other sites, including Tyr570, occurs only upon cytokine stimulation and activation of JAK2 Feener, et al., Mol. Cell. Biol. 2004; 24: 4968-4978; Argetsinger, et al., Mol. Cell. Biol. 2004; 24: 4955-4967). The kinases responsible for phosphorylation of Ser523 and Tyr570 have not been identified, but the activity of JH1 was not required for these phosphorylation events. (Ishida-Takahashi, et al., Mol. Cell. Biol. 2006; 26: 4063-4073; Mazurkiewicz-Munoz, et al., Mol. Cell. Biol. 2006; 26: 4052-4062; Feener, et al., Mol. Cell. Biol. 2004; 24: 4968-4978; Argetsinger, et al., Mol. Cell. Biol. 2004; 24: 4955-4967). We show here that JH2 phosphorylates Ser523 and Tyr570, and that autophosphorylation of Ser523 is the primary event in JH2 activation and it is observed in unstimulated conditions (FIG. 14d). Cytokine-induced receptor dimerization and juxtapositioning of the JAKs lead to other regulatory trans-phosphorylation events, including phosphorylation of Tyr570. Ser523 resides in the linker region between the SH2-like domain of JAK2 and JH2, and from steric considerations could be phosphorylated in cis. Tyr570, predicted to be in the □2-□3 loop of JH2, is distal to the JH2 active site and is presumed to be phosphorylated in trans by JH2 in another JAK2 molecule. A crystal structure of JH1-JH2 will be required to understand the mechanisms by which JH2 sterically inhibits JH1 and by which JH2-mediated phosphorylation of Ser523 and Tyr570 suppresses JH1 activity. Our results are consistent with a model whereby phosphorylation of Ser523 and Tyr570 strengthens the JH1-JH2 autoinhibitory interaction. The relatively low catalytic activity of JH2 is in accordance with autophosphorylation of regulatory residues as a physiological function for JH2 while the JH1 is mainly responsible for phosphorylation of substrate proteins. The low catalytic activity, together with the critical regulatory role of Ser523 and atypical requirement for Mn2⁺ for catalysis, have probably hampered the detection of JH2 activity.

The discovery of somatic mutations in JH2 of JAK2 in the majority of Philadelphia chromosome-negative MPNs and other hematological malignancies have focused attention on the functional role of JH2 and turned JAKs into important therapeutic targets. Several inhibitors targeting the JAK2 tyrosine kinase domain are in clinical trials for MPNs (Santos, et al., Blood Rev. 2010). The JAK2 inhibitors show beneficial clinical effects and alleviate symptoms, but they do not substantially reduce the JAK2-mutant tumor load, and the inhibitors do not discriminate between normal and mutated JAK2. These data show that MPN-causing JAK2 mutations disturb JH2 catalytic activity and remove the negative regulatory effects of Ser523 and Tyr570 phosphorylation in cell lines and in primary cells from MPN patients. These results identify a molecular pathogenic mechanism in MPNs and suggest that loss of JH2 function is involved in the hyperactive JAK2 MPN phenotype.

In conclusion, these data demonstrate an unexpected regulatory mechanism for JH2 in JAK2. JH2 is an active protein kinase that autophosphorylates two negative regulatory residues, which is required to maintain a low-activity level of JAK2 in the absence of cytokine stimulation. The discovery of JH2 catalytic activity and its connection to MPNs provides novel approaches for the development of targeted therapies to combat JAK-mediated diseases.

### Example 4

### Materials and Methods

Protein expression, purification and crystallization. We expressed human Jak2 JH2 (residues 536-812), wild-type (JH2-WT) and V617F (JH2-VF), with a C-terminal thrombin-cleavable His₆-tag in baculovirus-infected Spodoptera frugiperda-9 cells. JH2-WT harbored the designed mutations W659A, W777A and F794H (described in Results). JH2-VF harbored W777A and F794H, but due to a cloning oversight contained native Trp659. We resuspended cell pellets in lysis buffer containing 20 mM Tris-HCl (pH 8.5), 500 mM NaCl, 10% (v/v) glycerol, 0.5 mM TCEP, 1 mM sodium orthovanadate and 20 mM imidazole, supplemented with protease inhibitors (Roche Diagnostics). We lysed the cells using a cell disruptor (Avestin), clarified the lysate by centrifugation and purified the protein by nickel-affinity chromatography (Ni-NTA, QIAGEN) and anion-exchange chromatography (Source-Q, GE Healthcare). JH2 eluted as a single peak on the Source-Q column, and fractions corresponding to the peak were concentrated to ˜6 mg ml⁻¹ using Amicon centrifugal filters (Millipore). Crystals of JH2-WT and JH2-VF, apo or with Mg-ATP (3 mM MgCl₂, 1 mM ATP), grew in hanging drops at 4° C. in 200 mM sodium acetate, 100 mM Tris-HCl (pH 8.0 or 8.5) and 18-21% (w/v) polyethylene glycol (PEG) 4000. Macroseeding was required to grow single crystals of sufficient size.

X-Ray Data Collection, Structure Determination and Refinement.

We collected diffraction data for apo JH2-WT (see Table 1 for unit-cell information) on a MicroMax-007 x-ray generator (Rigaku) (λ=1.5418 Å) and an RAXIS IV++ image plate detector (Rigaku). We collected data for ATP-bound JH2-WT and JH2-VF at beam line X25 (λ=0.9789 Å) at the National Synchrotron Light Source, Brookhaven National Laboratory on a Pilatus 6M CCD detector (DECTRIS). We processed the data using HKL-2000 (Otwinowski, et al., Methods Enzymol., 1997; 276: 307-326). We determined the structure of apo JH2-WT by molecular replacement (one molecule in the asymmetric unit), trying many tyrosine kinase structures (separate N and C lobes or both lobes) as search models. Ultimately, we were successful in placing the C lobe of JH2-WT with MOLREP (Vagin, et al., J. Appl. Cryst., 1997; 30: 1022-1025), using as a search model the C lobe (poly-alanine) of the epidermal growth factor receptor (EGFR) kinase domain (PDB code 1M14). We were unable to obtain a solution for the N lobe of JH2-WT (with the C lobe fixed) by molecular replacement, and manually placed the N lobe (poly-alanine) of the EGFR kinase domain in the (weak) 2Fₒ-F_{C} electron-density map generated from the JH2-WT C-lobe model, followed by rigid-body refinement in REFMAC (Murshudov, et al., Acta Crystallogr. D, 1997; 53: 240-255). Iterative rounds of model-building in Coot (Emsley, et al., Acta Crystallogr. D, 2004; 60: 2126-2132) and positional and B-factor refinement (with TLS (Painter, et al., Acta Crystallogr. D Biol. Crystallogr., 2006; 62: 439-450) with REFMAC led to an atomic model of apo JH2-WT at 2.0-Å resolution that includes all residues from 536 to 810. We determined the crystal structures of JH2-WT and JH2-VF with bound Mg-ATP by molecular replacement using the JH2-WT apo structure as the search model.

Molecular Dynamics Simulations.

For the starting structures in the simulations, the mutations introduced to increase protein solubility (see above) were reverted back to their wild-type residues. Simulation systems were set up by placing the protein at the center of a cubic simulation box (with periodic boundary conditions) of at least 70 Å per side. Explicitly represented water molecules were added to fill the system, and Na⁺ and Cl⁻ ions were added to maintain physiological salinity (150 mM) and to obtain a neutral total charge for the system. The systems were parameterized using the Amber ff99SB-ILDN force field with TIP3P water (Hornak, et al., Proteins, 2006; 65: 712-725; Lindorff-Larsen, et al., Proteins, 2010, 78: 1950-1958 and Jorgensen, et al., J. Chem. Phys., 1983; 79: 926-935). Equilibrium molecular dynamic simulations were performed on the special-purpose molecular dynamics machine Anton (Shaw, et al., In ACAM/IEEE Conference on Supercomputing (New York, N.Y., ACM Press), 2009) in the NVT ensemble at 310 K using the Nose-Hoover thermostat (Hoover, Phys. Rev. A, 1985; 31: 1695-1697) with a relaxation time of 1.0 ps and a time step of 2.5 fs. All bond lengths to hydrogen atoms were constrained using a recently developed implementation (Lippert, et al., J. Chem. Phys., 2007; 126: 046101) of M-SHAKE Krautler, et al., J. Comput. Chem., 2001, 22: 501-508). The Lennard-Jones and the Coulomb interactions in the simulations were calculated using a force-shifted cutoff of 12 Å (Fennell, et al., J. Chem. Phys., 2006; 124: 234104).

In Vitro Autophosphorylation of JH2.

We produced and purified human Jak2 JH2, residues 513-824, as described previously (Ungureanu, et al., Nat. Struct. Mol. Biol., 2011, 18: 971-976), isolating two peaks from the Mono-Q (GE Healthcare) column: non-phosphorylated and Ser523-phosphorylated JH2. The autophosphorylation reaction buffer contained 10 mM ATP, 10 μCi [γ-³²P] ATP (PerkinElmer), 20 mM Tris-HCl (pH 8.0), 20 mM MnCl₂, 300 mM NaCl, 10% (v/v) glycerol and 0.5 mM TCEP. We performed the reactions at room temperature and stopped them by adding 2×SDS-PAGE sample buffer.

Cell Transfection and Western Blotting.

Transfection of HA-tagged human Jak2, wild-type or mutants (obtained by QuikChange (Stratagene)), into Jak2-deficient γ2A cells has been described (Ungureanu, et al., Nat. Struct. Mol. Biol., 2011, 18: 971-976). We used the following antibodies for immunoprecipitation or Western blotting: anti-HA (Covance), anti-Jak2 pY1007-1008 (Cell Signaling Technology) and anti-Jak2 pSer523 (Ishida-Takahashi, et al., Mol. Cell. Biol., 2006; 26: 4063-4073).

TABLE 1
X-ray data collection and refinement statistics

| | | |
|---|---|---|
| JH2-WT | JH2-WT | JH2-VF |
| (apo) | (Mg-ATP) | (Mg-ATP) |

Data collection

| | | | |
|---|---|---|---|
| Space group | P2₁ | P2₁ | P2₁ |
| Cell | | | |
| dimensions | | | |
| a, b, c (Å) | 44.4, 57.1, 61.0 | 44.6, 57.5, 60.9 | 46.9, 57.3, 60.5 |
| αβγ (°) | 90.0, 110.4, 90.0 | 90.0, 110.8, 90.0 | 90.0, 111.8, 90.0 |
| Resolution (Å) | 50.0-2.0 | 50.0-1.75 | 50.0-2.0 |
| R_{sym} or R_{merge} | 3.9 (12.1) | 6.6 (43.1) | 5.5 (21.5) |
| I/σI | 37.3 (12.1) | 25.5 (2.6) | 19.1 (2.8) |
| Complete- | 99.6 (99.4) | 99.2 (94.1) | 95.3 (66.4) |
| ness (%) | | | |
| Redundancy | 3.7 | 6.4 | 3.5 |

Refinement

| | | | |
|---|---|---|---|
| Resolution (Å) | 50.0-2.0 | 50.0-1.75 | 50.0-2.0 |
| No. reflections | 18,109 | 27,543 | 18,524 |
| R_{work}/R_{free} | 18.1/21.7 | 17.8/20.6 | 18.1/22.8 |
| No. atoms | | | |
| Protein | 2,145 | 2,133 | 2,144 |
| Ligand/ion | 12 | 42 | 34 |
| Water | 152 | 147 | 111 |
| B-factors | | | |
| Protein | 31.9 | 32.7 | 39.6 |
| Solvent | 39.6 | 39.3 | 41.8 |
| r.m.s | | | |
| deviations | | | |
| Bond | 0.007 | 0.009 | 0.008 |
| lengths (Å) | | | |
| Bond | 1.2 | 1.4 | 1.3 |
| angles (°) | | | |

One crystal was used per data set. Values in parentheses are for highest-resolution shell.

### Results.

Crystal Structure of Jak2 JH2.

We initially engineered two baculoviruses to encode human Jak2 JH2, residues 536-827, both wild-type and V617F. We expressed these two proteins in soluble form in insect cells, but the proteins showed signs of aggregation during purification and did not yield crystals. Based on our homology model of Jak2 JH2, we identified three putative solvent-exposed hydrophobic residues (all in the C lobe) for possible substitution: Trp659, Trp777 and Phe794 (each of which is not conserved in Jaks). Full-length Jak2 bearing the mutations W659A, W777A and F794H was phosphorylated in cells to the same extent as wild-type (data not shown). The mutated versions of wild-type and V617F JH2 (see Methods for details), also with a new C-terminus at residue 812, expressed at higher levels, were better behaved during purification and readily yielded crystals. These proteins will hereafter be referred to simply as JH2-WT (wild-type) and JH2-VF (V617F).

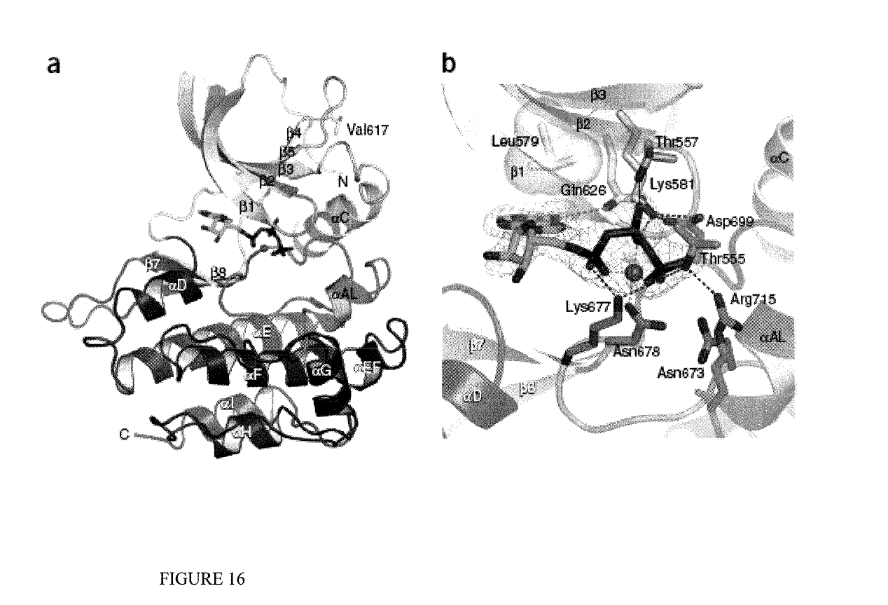

We obtained crystal structures of JH2-WT without nucleotide (apo; 2.0-Å resolution) and with Mg-ATP bound (co-crystallization; 1.75-Å resolution), each with one JH2 molecule per asymmetric unit. Data collection and refinement statistics appear in Table 1. Jak2 JH2 adopts the prototypical serine-threonine and tyrosine kinase fold, with an N lobe comprising a five-stranded β sheet and one a helix (αC), and a C lobe that is mainly α-helical (FIG. 16a). The domain structure of JH2 begins at Phe537 and ends at Leu808, residues that are conserved in JH2 of Jaks. Notable overall features of the JH2 structure include a relatively short (non-phosphorylatable) activation loop (seven residues shorter than in Jak2 JH1), which terminates in an α helix, and an extended loop between β-strand 7 (β7) and β8 (eight residues longer than the corresponding loop in JH1).

JH2s of Jaks lacks several residues that, in canonical protein kinases, are important for catalysis. The catalytic loop in canonical protein kinases contains a conserved aspartate that plays a key role in the phosphoryl transfer reaction. In Jak JH2s, an asparagine (Asn673 in Jak2) replaces the aspartate. The activation loop of canonical protein kinases begins with a DFG sequence motif, whereas Jak JH2s contain DPG. Finally, in canonical protein kinases, a conserved lysine in (33 (Lys581 in Jak2) is salt-bridged to a conserved glutamate in αC, the latter of which is replaced by alanine or threonine in Jak JH2s (Ala597 in Jak2).

ATP binding mode and comparison with other protein kinases. As in canonical protein kinases, Mg-ATP binds in the cleft between the N and C lobes of Jak2 JH2 (FIG. 16a,b). The structures of JH2-WT, with and without bound Mg-ATP, are very similar, with a root-mean-square deviation (r.m.s.d.) in Cα positions (residues 537-808) of only 0.44 Å. The apo structure exhibits a closed lobe configuration, which is not appreciably altered upon Mg-ATP binding. This trait is probably due in part to a C lobe-N lobe contact mediated by conserved (Jak family) Arg715 at the end of the activation loop, which in both the apo and ATP-bound forms of JH2 makes a hydrogen bond with Thr555 in the nucleotide-binding loop (FIG. 16b). One salient difference between the apo and Mg-ATP-bound structures is found in αC. In the apo structure, αC is disrupted midway by an intercalating water molecule that bridges backbone atoms Phe594(O) and Ala598(N) in the helix. Binding of Mg-ATP displaces this water molecule, but the backbone hydrogen-bonding in αC remains irregular.

There are several features that distinguish the ATP-binding mode in JH2 from that in canonical protein kinases (FIG. 16b). In JH2, the residue from the N lobe that flanks the adenine base is Leu579 (β3), whereas alanine is highly conserved at this position (VA(V/I)K) motif) in canonical protein kinases. Two threonine residues in the nucleotide-binding loop of JH2 are hydrogen-bonded to ATP phosphate groups: Thr555 with the γ phosphate and Thr557 with the γ phosphate. Thr557 is typically a glycine in canonical protein kinases (GXGXXG motif, where X is any amino acid). The so-called gatekeeper residue in the back of the ATP-binding cleft is typically hydrophobic, but in JH2 is a glutamine (Gln626), which is hydrogen-bonded to the adenine base and to Asp699 (DPG) in the activation loop.

A single Mg²⁺ ion is present in the structure, which is coordinated by Asn678 (catalytic loop), an oxygen atom from each of the three ATP phosphate groups and one water molecule (FIG. 16b). In canonical protein kinases such as (serine-threonine) protein kinase A (PKA) (Zheng, et al., Acta Cryst., 1993; D49: 362-365) or the insulin receptor tyrosine kinase (Hubbard, EMBO J., 1997; 16: 5572-5581), two Mg²⁺ ions are present, and the aspartate of the DFG motif coordinates both ions. Rather than coordinating a Mg²⁺ ion, Asp699 in JH2 (DPG) is salt-bridged to Lys581, the conserved β3 lysine. This interaction evidently substitutes for the canonical β3 lysine-αC glutamate salt bridge. A superposition of the active sites of JH2 and PKA reveals that the γ phosphate of ATP in the JH2 structure is positioned for phosphoryl transfer.

JH2s of Jaks shares several sequence characteristics with Her3 (ErbB3), a member of the epidermal growth factor receptor family. Her3 was also characterized as a pseudokinase, but was recently shown to possess weak catalytic activity (Shi, et al., Proc. Natl. Acad. Sci. USA., 2010; 107:7692-7697). In both kinases, the conserved glutamate in αC is absent, and asparagine replaces aspartate in the catalytic loop. In the Her3 structure (Shi, et al., Proc. Natl. Acad. Sci. USA., 2010; 107:7692-7697 and Jura, et al., Proc. Natl. Acad. Sci. U.S.A., 2009; 106: 21608-21613), Asp833 from the DFG motif is salt-bridged to the β3 lysine (Lys723), similar to Asp699 in Jak2 JH2, but it also coordinates the lone Mg²⁺ ion. The γ phosphate of the nucleotide is in a different position in the two structures, which could be due to co-crystallization with AMPPNP (Her3) versus ATP (Jak2).

Cis- Versus Trans-Autophosphorylation of Ser523 and Tyr570.

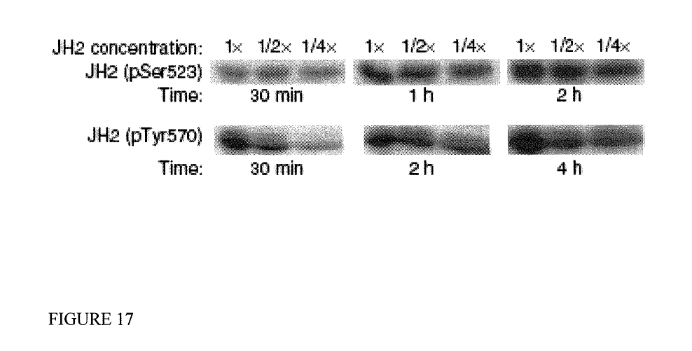

We determined whether the two sites we had identified previously (Ungureanu, et al., Nat. Struct. Mol. Biol., 2011, 18: 971-976), Ser523 and Tyr570, are autophosphorylated via a cis- or trans-mechanism. Simple modeling based on the JH2 crystal structure suggests that Ser523, in the SH2-JH2 linker, could possibly reach the JH2 active site to be autophosphorylated in cis, whereas Tyr570 in the β2-β3 loop of JH2, far removed from the active site, would necessarily be autophosphorylated in trans. As described previously (Ungureanu, et al., Nat. Struct. Mol. Biol., 2011, 18: 971-976), we purified a longer form of JH2 (residues 513-827, which includes Ser523) by anion-exchange chromatography, which yielded two JH2 peaks representing unphosphorylated and Ser523-phosphorylated JH2. We incubated unphosphorylated JH2 with Mn[γ-³²P] ATP at several different protein concentrations and for several time points. The data show that the phosphorylation level of Ser523 is independent of JH2 concentration, consistent with autophosphorylation in cis (FIG. 17, top). Moreover, these data provide further proof, beyond expression of a catalytically inactive JH2 mutant (K581A) (Ungureanu, et al., Nat. Struct. Mol. Biol., 2011, 18: 971-976), that Ser523 is phosphorylated by JH2 and not by a contaminating protein kinase, which would necessarily be concentration-dependent. Similarly, we performed an autophosphorylation reaction with Ser523-phosphorylated JH2 (from the second ion-exchange peak). In this case, the phosphorylation level of Tyr570 was concentration-dependent, consistent with autophosphorylation in trans (FIG. 17, bottom).

Crystal Structure of Jak2 JH2 V617F.

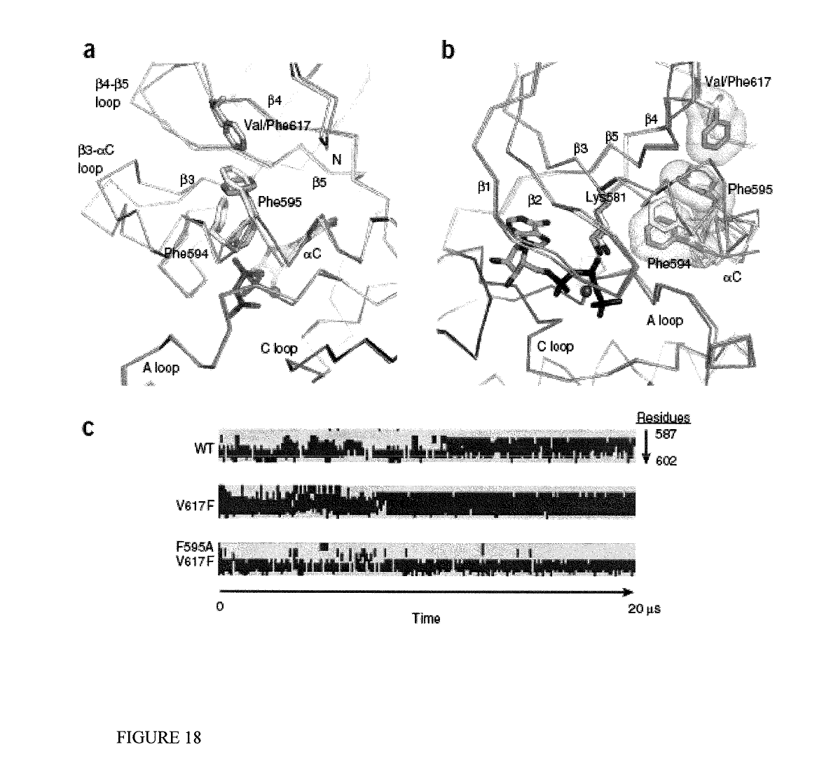

We obtained a crystal structure at 2.0-Å resolution of the pathogenic JH2 mutant, V617F (JH2-VF), with Mg-ATP bound, in the same monoclinic lattice as JH2-WT (Table 1). Val617 is situated in the β4-β5 loop in the N lobe of JH2 (FIG. 16a). Overall, the structures of JH2-VF and JH2-WT are highly similar, with an r.m.s.d. in Cα positions (residues 537-808) of 0.76 Å (FIG. 18a). The mode of nucleotide binding to JH2-VF is indistinguishable from that of JH2-WT. Substantive structural deviations between JH2-VF and JH2-WT occur in αC and in the β3-αC and β4-β5 loops. Most notably, in contrast to its distorted structure in JH2-WT, αC in JH2-VF exhibits continuous backbone hydrogen-bonding and is extended by an additional turn on the N-terminal end. Phe617 (replacing valine) causes a rotation in the phenyl ring of Phe595 (αC) and induces a major shift in the side-chain position of neighboring Phe594 (FIG. 18a). Phe617, Phe595 and Phe594 form π-stacking interactions (T-shaped) (FIG. 18b), with a closest inter-ring carbon-carbon distance of 3.8 Å for both Phe617-Phe595 and Phe595-Phe594. The Phe594 perturbation alters slightly the side-chain position of Lys581 (FIG. 18b), which might explain why the catalytic activity of JH2 is impaired in V617F (Ungureanu, et al., Nat. Struct. Mol. Biol., 2011, 18: 971-976).

Molecular Dynamics Simulations.

To explore further the structural differences between JH2-WT and JH2-VF and the relative stability of αC, we performed long-scale (˜20 μs) Molecular dynamics simulations of the two proteins in their apo forms. The simulations show that αC in wild-type JH2 is prone to melting and that Phe617 stabilizes the helix (FIG. 18c), corroborating the crystallographic data. Stabilization of αC is likely due in part to the □-stacking interactions between Phe617, Phe595 and Phe594. However, during the 20-μs simulation, these three residues are in contact only transiently, suggesting that Phe617 stabilizes □C through indirect mechanisms as well. The simulations also indicate that, overall, JH2 is intrinsically flexible in comparison with other protein kinases. The catalytic loop is one of the more stable polypeptide segments in protein kinases and, over the simulation, the r.m.s.d. in C-α positions for the catalytic loop of JH2-WT (residues 671-678) was 1.99 Å, versus 0.76 Å for the same eight residues in the catalytic loop of the Src tyrosine kinase (residues 384-391). This is probably due, at least in part, to a glycine in the JH2 catalytic loop (Gly672 in Jak2) in place of the canonical arginine (HRD motif). A small amino acid is required at this position to accommodate the shorter activation loop of JH2 and its divergent conformation.

Effects of JH2 Mutations in Full-Length Jak2.

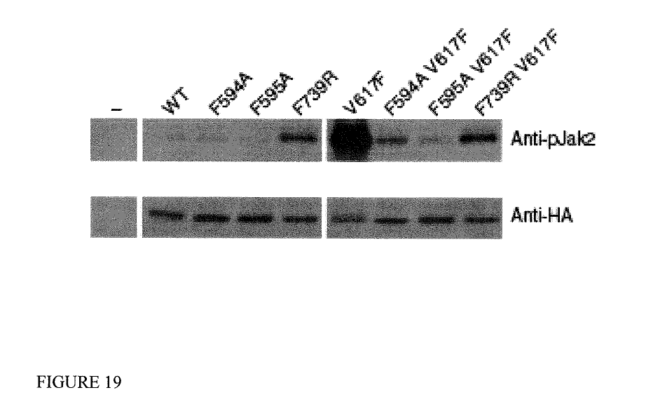

To probe the importance of Phe594 and Phe595 in αC for the constitutive activity of V617F, we introduced the point mutations F594A or F595A into either HA-tagged wild-type Jak2 or V617F (double mutants) and expressed the proteins in γ2A mammalian cells that lack endogenous Jak2. As observed previously, the basal (non-cytokine-stimulated) activation state of Jak2 V617F, as measured by JH1 activation-loop phosphorylation (pY1007, pY1008), is markedly enhanced compared to wild-type Jak2 (FIG. 19). The single point mutations in αC (F594A or F595A) did not substantially change the basal activation state of Jak2, but they caused a dramatic loss of constitutive activity of V617F (FIG. 19) (Dusa, et al., PLoS ONE, 2010; 5: e11157 and Gnanasambandan, et al., Biochemistry, 2010; 49: 9972-9984). To investigate whether destabilization of αC by F595A could account for the loss of V617F activity, we performed molecular dynamic simulations on the double mutant, F595A V617F. Indeed, F595A caused a reversion of αC stability back to the wild-type level, if not below (FIG. 18c). Because F595A suppresses not only the hyperactivity of (proximal) V617F, but also of R683G (β7-β8 loop), and even of T875N in JH1 (ref 24), it suggests that F595A intrinsically destabilizes αC and that the structural integrity of αC is critical for the stimulatory interaction mediated by JH2.

Because destabilization of αC in the N lobe was found to suppress the constitutive activity of V617F, we asked what effect destabilization of the C lobe would have on Jak2 activity. For this purpose, we introduced the mutation F739R. Phe739 in aF is buried in the hydrophobic core of the C lobe, and mutation to arginine should severely destabilize the C lobe. In contrast to F594A and F595A, F739R showed a marked increase in basal phosphorylation of Jak2 in γ2A cells (FIG. 19). Similar to F594A and F595A, but to a lesser extent, F739R suppressed the activity of V617F. The behavior of F739R largely mimics that of the Jak2 JH2 deletion mutant (Saharinen, et al., J. Biol. Chem., 2002; 277: 47954-47963)—increased but sub-maximal Jak2 activity—and, from the comparison with F595A (no increase in basal activity, major suppression of V617F activity), suggests that the C lobe plays a more important role in the JH2-mediated inhibitory interaction than it does in the stimulatory interaction.

1. A method for identifying an agent capable of binding to a Janus kinase Jak2 V617F comprising:
(a) determining an ability of the agent to fit into a three-dimensional structure formed by the Jak2 V617F; and
(b) selecting a test compound predicted to fit the three-dimensional structure.

2. The method of claim 1, wherein the method is computer-assisted.

3. The method of claim 2, wherein the computer-assisted method comprises virtual ligand docking and screening techniques capable of designing and/or identifying the agent predicted to bind the three-dimensional structure of the Jak2 V617F.

4. The method of claim 3, wherein the compound predicted to bind to the three-dimensional structure is predicted to bind with high affinity.

5. The method of claim 1, wherein the agent inhibits an activity of a Jak.

6. The method of claim 1 wherein the agent is a small molecule.

7. The method of claim 1 wherein the agent binds preferentially to the Jak2 V617F.

8. A method for identifying an agent capable of selectively binding to a Janus kinase Jak2 V617F comprising:
(a) providing a Jak JH2 wild type and a Jak2 JH2 V617F protein; and
(b) identifying the agent that binds to the Jak2 JH2 V617F but does not substantially bind to the Jak JH2.

9. The method of claim 8, wherein the agent binds the Jak2 JH2 V617F with high affinity.

10. The method of claim 8, wherein the agent inhibits an activity of a Jak.

11. The method of claim 8 wherein the agent is a small molecule.

12. An agent capable of selectively binding to a Jak2 V617F.

13. An agent identified by the method of claim 1 or claim 8.

14. A method for treating a myeloproliferative neoplasia (MPN) comprising administering an agent that selectively inhibits a Jak2 V617F.

15. The method of claim 14 wherein the myeloproliferative neoplasia (MPN) is selected from the group consisting of polycythemia vera, primary myelofibrosis, essential thrombocythemia, acute lymphoblastic leukemia, and acute myeloid leukemia.

16. The method of claim 14 wherein the agent is a small molecule.
